(19) United States
(12) Patent Application Publication
(10) Pub. No.: US 2024/0301386 A1
(43) Pub. Date: Sep. 12, 2024

(54) Soluble Guanylyl Cyclase (GC1) Mimetic Polypeptides and Methods of Use Thereof

(71) Applicant: Rutgers, the State University of New Jersey, New Brunswick, NJ (US)

(72) Inventor: Annie V. Beuve, Bloomfield, NJ (US)

(73) Assignee: Rutgers, the State University of New Jersey, New Brunswick, NJ (US)

(21) Appl. No.: 18/597,329

(22) Filed: Mar. 6, 2024

Related U.S. Application Data
(60) Provisional application No. 63/451,158, filed on Mar. 9, 2023.

(51) Int. Cl. C12N 9/88; A61K 38/00; A61K 47/64; A61P 35/00

(52) U.S. Cl. CPC C12N 9/88; A61K 47/64; A61P 35/00; A61K 38/00; C12Y 406/01002

(57) ABSTRACT

The present disclosure relates, in part, to soluble guanylyl cyclase (GC1) mimetic polypeptides comprising amino acid residues 581-635 of the α-subunit of soluble guanylyl cyclase (GC1), or a fragment thereof, and at least one cell penetrating peptide (CPP). The present disclosure further relates to methods of treating, preventing, and/or ameliorating a disease or disorder in a subject, including but not limited to cancer, hypertension, hypotension, and cardiac dysfunction. In certain embodiments, the methods comprising administering to the subject the polypeptide of the present disclosure.

### CROSS-REFERENCE TO RELATED APPLICATIONS

The present application claims priority under 35 U.S.C. § 119(e) to U.S. Provisional Patent Application No. 63/451,158 filed Mar. 9, 2023, which is incorporated herein by reference in its entirety.

### STATEMENT REGARDING FEDERALLY SPONSORED RESEARCH AND DEVELOPMENT

This invention was made with government support under GM067640 and GM112415 awarded by the National Institutes of Health. The government has certain rights in the invention.

### SEQUENCE LISTING

This application contains a Sequence Listing which has been submitted electronically in ASCII format and is hereby incorporated by reference in its entirety. The ASCII copy, created on Feb. 24, 2024, is named “370602-7068US1_Sequence_Listing.xml” and is 32,346 bytes in size.

### BACKGROUND

Soluble guanylyl cyclase (GC1) and the oxido-reductase thioredoxin (Trx1) form a complex that mediates two NO signaling pathways as a function of the redox state of the cells. Under physiological conditions, reduced Trx1 (rTrx1) supports the canonical NO-GC1-cGMP pathway, by protecting GC1 activity from oxidation of thiols. Under oxidative stress, the NO-cGMP pathway is disrupted by S-nitrosation of GC1 (i.e., addition of a NO group to a cysteine of GC1). In turn, SNO-GC1 initiates transnitrosation cascades, using oxidized thioredoxin (oTrx1) as nitrosothiol relay.

GC1 and Trx1 physically interact under conditions of oxidative stress to form a GC1/Trx1 complex which modulates the activity of caspase-3. Caspase-3, which is known as an executioner caspase, plays a critical role in the process of apoptosis, inducing cell death while in an active form. Interaction of the GC1/Trx1 complex and caspase-3 renders the caspase inactive, thereby at least partially inhibiting apoptosis, resulting in increased and/or aberrant cellular proliferation which characterizes tumor growth. Further, GC1/Trx1 complex formation also occurs in blood vessels. Under homeostatic conditions, the GC1/Trx1 complex regulates vasodilation, while altering vasodilation under oxidative stress. Further, vasodilation is an important mechanism in the regulation of blood pressure.

There is thus a need in the art for compositions useful for inhibiting the interaction and/or complex formation of GC1 and Trx1, and methods of use thereof. The present disclosure addresses this need.

### SUMMARY

The disclosure provides in one aspect a construct comprising a polypeptide comprising amino acid residues 581-635 of soluble guanylyl cyclase (GC1) (SEQ ID NO:34), SVFAGVVGGKMPRYCLFGNNVTLANKFESCSVPRKINVSPTTYRLLKDCPGFVFT (SEQ ID NO:7), or a fragment thereof. In certain embodiments, the polypeptide is further conjugated to at least one cell penetrating peptide (CPP). In certain embodiments, the polypeptide is further derivatized.

The disclosure provides in another aspect a pharmaceutical composition comprising a construct provided herein and at least one pharmaceutically acceptable carrier.

The disclosure provides in yet another aspect a method of treating, preventing, and/or ameliorating a disease or disorder in a subject. In certain embodiments, the method comprises administering to the subject a therapeutically effective amount of a construct comprising a polypeptide comprising amino acid residues 581-635 of soluble guanylyl cyclase (GC1) (SEQ ID NO:34), SVFAGVVGGKMPRYCLFGNNVTLANKFESCSVPRKINVSPTTYRLLKDCPGFVFT (SEQ ID NO:7), or a fragment thereof.

### BRIEF DESCRIPTION OF THE FIGURES

The drawings illustrate generally, by way of example, but not by way of limitation, various embodiments of the present application.

FIG. 1 depicts the sequence of exemplary peptides designed to disrupt the GC1/Trx1 complex in a purified system. Peptides 1, 2 and 3 were designed in the proximity of C610. The C-terminal region of the GC1 α-subunit is shown (human sequence provided, which shares 100% identity with rat, mouse and bovine sequences in this region). Peptide 1: (601) VTLANKFESCS (611) (SEQ ID NO:1); Peptide 2: (605) NKFESCSVPRK (615) (SEQ ID NO:2); and Peptide 3: (614) RKINVSPTTYR (624) (SEQ ID NO:3). Peptide 4 (scramble): KSRVNPSKCEF (SEQ ID NO:4) (not shown) comprises an arbitrarily ordered homologous sequence of peptide 2.

FIGS. 2A-2D depict an exemplary peptide designed to disrupt the GC1/Trx1 complex in cells and a control. FIG. 2A provides exemplary peptide sequences for modified-peptide 3 (614-624) (SEQ ID NO:5) and modified-peptide 4 (i.e., scramble) (SEQ ID NO:6); peptide 3 (highlight) was further selected for addition of the fluorophore Fluorescein isothiocyanate (FITC) at the N-terminus followed by a cell penetrating sequence—RKKRWFRRRRPKWKK (SEQ ID NO:8). Scramble peptide (4) was similarly modified. FIG. 2B depicts imaging of peptides penetration in COS-7 cells. Imaging of COS-7 cells overexpressing GC1 treated with DMSO (0.7%) or 10 μM inhibitory peptide (3) or scramble peptide (4). Representative images taken with a Zeiss 200 microscope (20× objective; 800 ms exposure of green fluorescence imaging). FIG. 2C depicts Western blots of lysates from COS-7 cells transfected with GC1 and treated with DMSO or peptides, showing similar overexpression of GC1 α and β subunits, and endogenous expression of thioredoxin 1. In each lane, 40 μg of lysates were subjected to electrophoresis (12% SDS-pAGE) and probe with anti-α (1:1000, ThermoFisher), anti-β (1:1000, Cayman) and anti-Trx1 (1:500, Cell signaling) antibodies. FIG. 2D shows that basal activity in COS-7 cells overexpressing GC1 is unchanged by the peptides treatment. In contrast to the NO-stimulated GC1 activity (FIG. 3B), there was no significant difference for the basal specific activity treated with DMSO (control), inhibitory peptide and the scramble peptide. N=4 independent experiments in duplicate.

FIGS. 3A-3B show that exemplary inhibitory peptides block Trx1-dependent enhancement of NO-stimulated GC1 activity in an in vitro system (FIG. 3A) and in cells (FIG. 3B). FIG. 3A: purified GC1 and Trx1 (molar ratio 1:7) were mixed in the absence or presence of M of each peptide, GC1 activity was measured under basal (not shown) and stimulated (1 μM DEA-NO) conditions; specific activity in nmol·min⁻¹·mg⁻¹ (FIGS. 2A-2D) was normalized against GC1 activity in the absence of Trx1 and expressed as fold change; n=4 independent experiments, measured in duplicate; *, p<0.05. FIG. 3B: COS-7 cells were transfected with GC1 and treated with 10 μM of peptide 3 (i.e., inhibitory peptide) and scramble peptide (4) modified by addition of a cell-penetrating peptide and the fluorophore FITC (FIG. 2A). Peptide penetration and GC1 expression were verified by imaging and Western blotting (FIGS. 2B-2C). n=4 independent experiments, measured in duplicate; *, p<0.05. The inhibitory peptide had no effect under basal conditions (FIG. 2D).

FIG. 4 shows that NO-stimulated activity of GC1 is not affected by incubation with the peptides alone in the in vitro/purified systems. DEA-NO was the NO-donor, used at 1 μM final concentration. n=4 independent experiments done in duplicate. NO-stimulated activity of GC1 was compared to NO-stimulated GC1 activity in the presence of the various peptides in the same in vitro/purified system as above.

FIGS. 5A-5B depict representative Western blots showing reducing activity of rGC and rTrx1 toward oTrx1 and oGC1, respectively and blocking of these activities by the inhibitory peptide. FIG. 5A: purified GC1 and Trx1 were oxidized or reduced as described herein and the various oxidized and reduced forms were mixed (molar ratio 1:5) as indicated on the top of the blots (lanes 1-4), then separated by electrophoresis on a non-reducing 12% SDS-PAGE gel. After transfer the blots were probed with GC1 anti-α and anti-β (upper panel), and anti-Trx1 (lower panel) antibodies. Lane 5-8 are the oxidized and reduced forms of Trx1 and GC1, lanes 9 and 10 are untreated GC1 and Trx1. M.W. for Molecular Weight markers. n=3 independent experiments. FIG. 5B: the oxidized and reduced forms of Trx1 and GC1 were mixed as above in the presence of DMSO (lane 1 and 4), 125 μM inhibitory peptide (inh-pep, lanes 2 & 5) or scramble peptide (scr-pep, lanes 3 & 6), then transferred and probed as in FIG. 5A. Lanes 7-8 are controls of reduced or oxidized forms of GC1 and Trx1 mixed together; n=3 independent experiments.

FIG. 6 depicts a Western blot under reducing conditions of the inputs used in FIG. 5A showing that the starting material was similar in each combination. Samples were electrophorated on 12% SDS-PAGE reducing gel, then transferred and probe with anti-α (1:1000, ThermoFisher), anti-β (1:1000, Cayman) and anti-Trx1 (1:500, Cell signaling) antibodies. The system of detection was an Odyssey imager, using fluorescent secondary antibodies.

FIGS. 7A-7B depict representative Western blots showing blunted transfer of SNO from GC1 to oTrx1 by the inhibitory peptide. FIG. 7A: purified GC1 was S-nitrosated with GSNO (SNO-GC1, lane 5); purified Trx1 was oxidized and mixed with SNO-GC1 in the presence of DMSO (lane 1), the inhibitory peptide (lane 2) or the scramble peptide (lane 3). Transfer of SNO from GC1 to oTrx1 (Lane 1, DMSO) was blunted by the inhibitory peptide (Lane 2), but maintained with the scramble peptide (Lane 3). Lane 4 shows that SNO-GC1 does not transnitrosate rTrx1 and lane 5 and 6 were S-nitrosated GC1 and oTrx1 alone. The samples were subjected to biotin switch assays then separated by electrophoresis on a non-reducing 12% SDS-PAGE gel. After transfer the blots were probed with anti-biotin antibodies (1:3000), to reveal S-nitrosated GC1 (upper panel) and S-nitrosated Trx1 (lower panel). The uncropped Western blots with the “no ascorbate” controls are provided in FIGS. 13A-13B. FIG. 7B: depicts Western blots of the starting materials used in FIG. 7A, run on a reducing 12% SDS-PAGE gel, then transferred and probed with anti-α, anti-β (upper panel), and anti-Trx1 (lower panel) antibodies. Note that in FIGS. 7A-7B, Trx1 migrates as a dimer and a monomer, respectively, and as expected with a non-reducing (FIG. 7A) vs. a reducing (FIG. 7B) gel electrophoresis.

FIGS. 8A-8B shows that, by reducing S-nitrosation in Jurkat T cells, an exemplary inhibitory peptide increases etoposide-dependent caspase-3 activity. FIG. 8A depicts representative Western blots of biotin switch assays of Jurkat T cells±etoposide (ETO) treated with DMSO, scramble peptide (Scr-pep) or inhibitory peptide (Inh-pep). Leftpanel is a blot of a non-reducing gel probed with anti-biotin showing the levels of S-nitrosation in Jurkat cells under the different conditions (left lane contains the M.W. markers). The S-nitrosation levels in Jurkat cells treated with the inhibitory polypeptide are drastically reduced. The right panel indicates the levels of S-nitrosated pro-caspase-3, cleaved caspase-3 and Trx1 after avidin purification of the biotinylated samples from the left panel. SNO-procasp-3 and SNO-Trx1 were remarkably reduced in cells treated with the inhibitory polypeptide (last 2 lanes); n=3; FIG. 8B: Caspase-3 activity was measured with the same sample's treatment of FIG. 8A. The experiments, measured in duplicate, were repeated independently 3 times. **, p<0.001; ***, p<0.0005. For clarity, the highly significant (p<0.0001) increase in apoptosis with ETO under the three conditions is not shown.

FIG. 9A: Ponceau red staining of the membrane of electrophorated samples on a 12% reducing gel, indicating similar protein amounts in the lysates of Jurkat T cells treated with DMSO±Etoposide (ETO), the scramble peptide±ETO, and the inhibitory peptide±ETO. FIG. 9B: depicts the blot of FIG. 9A probed with anti-alpha, anti-beta (Cayman, 1:1000), anti-casp-3 (Novus Biologicals, 1:500) and anti-Trx1 (Cell signaling, 1:1000) antibodies. Note the decreased intensity of procasp-3 signal suggesting increased processing in Jurkat cells treated with etoposide (ETO at 8 μM) and in the inhibitory peptide compared to the controls (DMSO, scramble peptide) treated with ETO.

FIGS. 10A-10B show samples of the biotin switch assays of FIG. 8A including samples not treated with ascorbate (−Asc) that were electrophorated on non-reducing gel. FIG. 10A: Ponceau red of the corresponding blot showing similar amount of proteins among of samples.

FIG. 10B: blot of FIG. 10A was probed with anti-biotin antibodies as described elsewhere herein.

FIG. 11 shows NO-stimulated specific activity of GC1±Trx1 mixed with each peptide, expressed in nmol/min·mg⁻¹. These data were used to calculate the fold changes and significance of difference between GC1 and the different mixtures of FIG. 3A. DEA-NO was the NO-donor, used at 1 μM final concentration. n=4 independent experiments in duplicate, expressed in nmol·min⁻¹·mg¹±S.E.M.

FIG. 12: Western blot under reducing conditions of the inputs used in FIG. 5B showing that the starting material was similar in each combination. Samples were electrophorated on 12% SDS-PAGE reducing gel, then transferred and probe with anti-α (1:1000, ThermoFisher), anti-β (1:1000, Cayman) and anti-Trx1 (1:500, Cell signaling) antibodies. The system of detection was an Odyssey imager, using fluorescent secondary antibodies.

FIGS. 13A-13B: Uncropped Western blots from FIG. 7A. Controls of the biotin switch assays were samples not treated with ascorbate (no Asc). FIG. 13A depicts imaging with fluorescent secondary antibodies using an Odyssey imager, and FIG. 13B depicts imaging without fluorescent secondary antibodies. Only GC1 was detectable in FIG. 13A; the blot was thus reprobed with HRP secondary antibodies and Trx1 was detected by enhanced chemiluminescence (ECL) (FIG. 13B).

FIGS. 14A-14C depict the mechanism of inhibition of proliferation by certain mimetic peptides of the present disclosure via increased apoptosis in Jurkat T cells (i.e., leukemic/lymphoma model). FIG. 14A depicts non-proliferative “normal” cells: the 3 molecules (i.e., GC1, Trx1, and caspase 3) are not connected and/or associated. FIG. 14B: upon oxidative stress (e.g., during tumor development), GC1, Trx1, and caspase 3 become attached and/or associated, caspase 3 is modified and becomes inactive, and cell death is inhibited leading to proliferation. FIG. 14C: addition of certain mimetic peptides to cells under oxidative stress or during tumor development results in failure of GC1 and Trx1 to attach, and GC1, Trx1, and caspase 3 are not connected and/or associated, caspase 3 is active, and cell death program proceeds.

FIGS. 15A-15B depict proposed models of the inhibitory effects of certain mimetic polypeptides of the present disclosure on NO-cGMP pathway signaling pathway and SNO transnitrosation cascades. FIG. 15A: under reducing conditions (e.g., in smooth muscle cells) rTrx1 and GC1 associate via formation of a mixed disulfide between C610 of the GC1 α subunit and C32 of Trx1 (i.e., C610 is endogenously S-nitrosated or/and engaged in a disulfide). In turn, Trx1 increases NO-stimulated GC1 activity, thus enhancing the NO-GC1-cGMP pathway. Addition of the inhibitory peptide, by blocking Trx1-GC1 complex formation, blunts Trx1 enhancement of NO-stimulated GC1 activity. FIG. 15B: oxidative and/or nitrosative stress induces multiple S-nitrosation of GC1 and oxidation of Trx1 (oTrx1) in Jurkat T cells, and the levels of S-nitrosation are endogenously elevated. A transnitrosation cascade, initiated by SNO-GC1 and mediated by oTrx1, leads to caspase-3 S-nitrosation, which renders it inactive leading to cell proliferation. In the presence of certain inhibitory polypeptides of the present disclosure, the SNO-GC1 and oTrx1 interaction is blocked, the transnitrosation cascade is disrupted, and thus caspase 3 remains active, supporting cell death.

FIG. 16 shows successful penetration of the inhibitory peptide in mesenteric artery (MA). The left panels indicate the bright field picture of two MA treated with DMSO (upper left) or the inhibitory peptide (lower left). Inverted fluorescent microscope was used to detect the FITC of the peptide (lower right panel) at 10× magnification (green fluorescence). The same exposure time was used for the DMSO and peptide-treated vessels. The yellow arrow indicates the lumen of the vessels; the orange arrow points to the endothelial cell layer and the white arrow to the smooth muscle cell layers.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, oncology, and peptide chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer, and the like. A tumor may be benign (benign tumor) or malignant (malignant tumor or cancer). Malignant tumors can be broadly classified into three major types. Malignant tumors arising from epithelial structures are called carcinomas, malignant tumors that originate from connective tissues such as muscle, cartilage, fat or bone are called sarcomas, and malignant tumors affecting hematopoietic structures (structures pertaining to the formation of blood cells) including components of the immune system, are called leukemias and lymphomas. Other tumors include, but are not limited to, neurofibromatosis.

The term “cell penetrating peptide” or “CPP” as used herein refers to short peptides that are capable of transporting different types of cargo molecules (e.g., polypeptides) across the plasma membrane, thus facilitating cellular uptake of various molecular cargoes. Cellular internalization of the cargo molecule bound to the cell penetrating peptide generally means transport of the cargo molecule across the plasma membrane and thus entry of the cargo molecule into the cell. Depending on the particular case, the cargo molecule can then be released in the cytoplasm, directed to an intracellular organelle, or also be presented on the cell surface. The cell-penetrating ability, or internalization, of the cell-penetrating peptide or the complex comprising said cell-penetrating peptide can be verified by standard methods known to one skilled in the art, including flow cytometry or microscopy of fluorescence of living and fixed cells, immunocytochemistry of cells transduced with said peptide or complex and Western blot.

The term “conjugated” as used herein refers to a covalent, non-covalent, or ionic bond that links one compound to another (e.g., two polypeptides). In certain embodiments, a cross-link may refer to a conjugation of one compound to another with a direct bond between the two compositions. In yet other embodiments, cross-link may refer to a conjugation which relies on a cross-linker or cross-linking agent. A cross-linker is a chemical species which forms a covalent or ionic bond to each of two compounds at opposing termini of the cross-linker, thereby cross-linking the two compounds without a direct covalent or ionic bond between the two compounds.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a compound are used interchangeably to refer to the amount of the compound that is sufficient to provide a beneficial effect to the subject to which the compound is administered.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound include, but are not limited to, intravenous, oral, aerosol, parenteral, ophthalmic, pulmonary and topical administration.

A “prophylactic” treatment is a treatment administered to a subject who does not exhibit signs of a disease or exhibits only early signs of the disease for the purpose of decreasing the risk of developing pathology associated with the disease.

The phrase “reduction of growth,” as used herein, refers to any reduced growth, replication rate, or colony formation exhibited by a neoplastic cell, a cancer cell, or a tumor in response to some therapeutic agent, treatment, or clinical intervention, such as radiation. For example, a neoplastic cell may exhibit a reduction in the cell's growth rate or its ability to replicate and form colonies in vitro or in vivo (e.g., when implanted as a tumor in an animal) in response to radiation.

The phrase “reduction in viability,” as used herein, refers to any reduction in survival exhibited by a neoplastic cell, a cancer cell, or a tumor in response to some chemotherapeutic agent, treatment, or clinical intervention, such as radiation. A neoplastic cell, a cancer cell, or a tumor may exhibit reduced viability in response to any such intervention by inhibition of progression of the cell through the cell cycle; damaged nucleic acids, proteins, or other macromolecules in a cell, induced terminal differentiation (senescence), in which the cell no longer replicates; inhibited cellular repair of nucleic acids; or increased rates of cell death by inducing apoptosis or “mitotic catastrophe”—a form of necrosis, when DNA damage levels are beyond those that can be effectively repaired.

The terms “sequence homology,” “percent identity (%),” “sequence identity,” “sequence identity percent,” or “percent identity” in the context of nucleotide and/or amino acid sequences refers to a quantitative measurement of the similarity between two sequences over an aligned region of the two sequences (e.g., two nucleotide sequences or two amino acid sequences).

A “therapeutic” treatment is a treatment administered to a subject who exhibits signs of pathology for the purpose of diminishing or eliminating those signs.

“Treating,” as used herein, means reducing the frequency with which symptoms are experienced by a patient or subject, or administering an agent or compound to reduce the severity with which symptoms are experienced by a patient or subject. An appropriate therapeutic amount in any individual case may be determined by one of ordinary skill in the art using routine experimentation.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Thioredoxin 1 (Trx1) is an oxido-reductase that was first described for its ability to reduce disulfide bonds and to protect against oxidative stress, through a mechanism that involves its reduced cysteine (Cys) active center (i.e., C³²xxC³⁵). As such, it is one of the most critical regulators of cellular redox and a key player of the antioxidant defense system in cells. Soluble guanylyl cyclase (GC1) forms a complex with Trx1, which in turn enhances NO-stimulated catalytic activity of GC1 in cells and in vitro.

The hundred-fold stimulation of cGMP production by NO requires the α and β subunits of GC1 to form a heterodimer. Trx1-trap system and mutational analysis suggested that a mixed disulfide between C³² of the Trx1 active site and Cys 610 (C⁶¹⁰) of the α subunit of GC1 was necessary for Trx1-dependent increase of GC1 response to NO stimulation.

NO-stimulated activity of GC1 can be reduced by S-nitrosation of several Cys residues. S-nitrosation (addition of a NO moiety to a Cys) is a posttranslational modification (PTM) that changes properties of the proteins including activity, localization and interaction. Interestingly, Trx1 can catalyze denitrosation, which is the removal of the nitroso group (SNO) from the Cys of a protein through a mechanism that involves its reduced C³²xxC³⁵ active center, as for disulfide reduction. As such, the mechanism of Trx1-dependent increase in GC1 activity could involve reduction of oxidized thiols and/or protection of GC1 from desensitization to NO by decreasing S-nitrosation of GC1.

Trx1 is involved in regulation of cellular S-nitrosation, not only via denitrosation but also via transnitrosation reactions. Transnitrosation by protein-protein interaction is the direct transfer of the nitroso group from the thiol on one protein to a target thiol on another protein, thus supporting a high degree of specificity for this PTM and its relevance as another form of NO signaling. Trx1 transnitrosation requires Trx1 to be oxidized (oTrx1) with formation of a disulfide bond between C³²—C³⁵ of its active site, while Cys73 (C⁷³) is the main SNO donor (i.e., transferring its SNO to the thiol of the targeted protein). It has recently been reported that the a subunit of GC1, in the absence of an active/cGMP-forming a/P heterodimer can itself transnitrosate more than 200 proteins under oxidative/nitrosative stress. Remarkably, this high number of GC1 SNO-targets is partially due to the ability of GC1 ability to interact with and use oTrx1 as a mediator of the S-nitrosothiols transfer.

It was found that the SNO-GC1→oTrx1 transnitrosation reaction was unilateral, and did not take place if Trx1 was in its reduced form (rTrx1) and that the SNO transfer involved C⁶¹¹ of the GC1-α subunit to C⁷³ of oTrx1. As example of the biological relevance of the transnitrosation reaction, a transnitrosation cascade initiated by SNO-GC1 that requires oTrx1 and leads to S-nitrosation of RhoA and inhibition of its activity in cells was specifically identified.

Together, these findings indicated that the GC1/Trx1 complex differentially supports NO signaling, that is cGMP production and nitrosation, as a function of the redox of the cells. In addition, it has been demonstrated that αC⁶¹⁰ is involved in both reduced and oxidized GC1/Trx1 complex formation and function. Previous studies used mutants of Trx1 or inhibitor of Trx1-reductase, thus altering the entire redox state of the cells and limiting the interpretation of the role of the GC1/Trx1 complex in cells. Likewise, these previous studies involved deletion of the α subunit of GC1 or replacement of Cys 610 with a Ser (α^{C610S}) with the caveat that such mutation could affect the structure and activity of GC1.

In order to (a) understand how a different GC1 and Trx1 interaction takes place under reduced and oxidative conditions; (b) characterize the complex properties; and (c) seek their biological relevance, a set of mimetic peptides of the αC610 region have been designed and prepared to disrupt the two complexes without affecting the integrity of Trx1 and GC1 under both reduced and oxidized conditions.

The present disclosure relates, in one aspect, to the discovery and development of an inhibitory peptide that blocks the interaction between GC1 and Trx1. This inhibition resulted in the loss of (a) rTrx1 enhancing effect of GC1 cGMP-forming activity in vitro and in cells and its ability to reduce the multimeric oxidized GC1; and (b) GC1 ability to fully reduce oTrx1, thus identifying GC1 novel reductase activity. Moreover, the inhibitory peptide blocked the transfer of S-nitrosothiols from SNO-GC1 to oTrx1.

In Jurkat T cells, oTrx1 transnitrosates procaspase-3, thereby inhibiting caspase-3 activity. Using an exemplary inhibitory peptide of the present disclosure, it is demonstrated herein that S-nitrosation of caspase-3 is the result of a transnitrosation cascade initiated by SNO-GC1 and mediated by oTrx1. Consequently, the peptide significantly increased caspase-3 activity in Jurkat cells, providing a promising therapy for one or more types of cancers.

### Polypeptide Compositions

In one aspect, the present disclosure provides a construct comprising a polypeptide comprising amino acid residues 581-635 of soluble guanylyl cyclase (GC1) (SEQ ID NO:34), or a fragment (contiguous segment) thereof. In certain embodiments, the polypeptide is further conjugated to at least one cell penetrating peptide (CPP).

In certain embodiments, the amino acid residues 581-635 of GC1 comprise SVFAGVVGGKMPRYCLFGNNVTLANKFESCSVPRKINVSPTTYRLLKDCPGFVFT (SEQ ID NO:7).

In certain embodiments, the polypeptide comprises a fragment of the amino acid residues 581-635 of soluble guanylyl cyclase (GC1). In certain embodiments, the fragment comprises 7, 8, 9, 10, 11, 12, 13, 14, 15, 16, 17, 18, 19, or 20 amino acid residues of the amino acid residues 581-635 of GC1.

In certain embodiments, the polypeptide comprises amino acid residues 601-635 of GC1, or a fragment thereof.

In certain embodiments, the fragment comprises 11 amino acid residues.

In certain embodiments, the N-terminus of the fragment corresponds to the amino acid residue 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, or 634 of GC-1.

In certain embodiments, the C-terminus of the fragment corresponds to the amino acid residue 582, 583, 584, 585, 586, 587, 588, 589, 590, 591, 592, 593, 594, 595, 596, 597, 598, 599, 600, 602, 603, 604, 605, 606, 607, 608, 609, 610, 611, 612, 613, 614, 615, 616, 617, 618, 619, 620, 621, 622, 623, 624, 624, 625, 626, 627, 628, 629, 630, 631, 632, 633, 634, or 635 of GC-1.

In certain embodiments, the fragment consists essentially of any one of SEQ ID NOs:1-3.

In certain embodiments, the fragment consists essentially of SEQ ID NO:3.

In certain embodiments, the at least one CPP shares at least 85%, 86%, 87%, 88%, 89%, 90%, 91%, 92%, 93%, 94%, 95%, 96%, 97%, 98%, 99%, or 99.5% sequence identity with any of SEQ ID NOs:8-33.

In certain embodiments, the at least one CPP comprises SEQ ID NO:8.

In certain embodiments, the amino acid residues 581-635 of GC1, or the fragment thereof, are covalently linked to the at least one CPP via the N-terminus amino group of the polypeptide, the C-terminus carboxyl group, and/or a nucleophilic moiety of an amino acid side chain.

In certain embodiments, the amino acid residues 581-635 of GC1, or the fragment thereof, are covalently linked to the CPP via the N-terminus amino group of the polypeptide.

In certain embodiments, the covalent linkage comprises an amide bond.

In certain embodiments, the CPP is conjugated to the polypeptide via a linker. In certain embodiments, the linker is any linker contemplated by one skilled in the art, such as a peptide comprising 1-50 independently selected amino acids, a polyethylene glycol group comprising 1-100 —(CH₂CH₂O)— groups, and/or a C₁-C₁₀₀ alkyl chain, for example.

In certain embodiments, the construct shares at least 85%, 86%, 87%, 88%, 89%, 90%, 91%, 92%, 93%, 94%, 95%, 96%, 97%, 98%, 99%, or 99.5% sequence identity with any of SEQ ID NO:5. In certain embodiments, the construct is SEQ ID NO:5.

In certain embodiments, at least one amino acid residue of the polypeptide is derivatized.

In certain embodiments, the derivatization comprises at least one selected from the group consisting of methylation, amidation, and acetylation. In certain embodiments, the derivatization comprises conjugation to a fluorophore. In certain embodiments, the fluorophore is fluorescein isothiocyanate (FITC).

### Cell Penetrating Polypeptides (CPPs)

Cell penetrating peptides typically have an amino acid composition that either contains a high relative abundance of positively charged amino acids such as lysine or arginine or has a sequence that contains an alternating pattern of polar/charged amino acids and hydrophobic nonpolar amino acids. These two types of structures are known as polycationic or amphipathic, respectively. Cell-penetrating peptides have different sizes, amino acid sequences, and charges, but all CPPs have one common feature, which is the ability to translocate the plasma membrane and facilitate delivery of various molecular charges to the cytoplasm or an organelle of a cell. Currently, CPP translocation theories distinguish three main mechanisms of entry: direct membrane penetration, endocytosis-mediated entry, and translocation by formation of a transient structure. The transduction of CPPs is an area of ongoing research. Cell-penetrating peptides have found numerous applications in medicine as drug delivery agents in the treatment of different diseases, including cancer and viral inhibitors, as well as contrast agents for cell labeling and imaging.

Typically, Cell Penetrating Peptides (CPP) are 8 to 50 residue peptides with the ability to cross the cell membrane and enter most cell types. Alternatively, they are also called a protein transduction domain (PTD), reflecting their origin as something that occurs in natural proteins. Frankel and Pabo, simultaneously with Green and Lowenstein, describe the ability of the transcriptional activator of human immunodeficiency virus 1 (HIV-TAT) trans-activation to penetrate cells (Frankel, A D and C O Pabo, Cellular uptake of the tat protein from human immunodeficiency virus. Cell, 1988. 55 (6): p. 1189-93). In 1991, the transduction in neuronal cells of the Antennapedia homeodomain (DNA-binding domain) of Drosophila melanogaster was described (Joliot, A., et al., Antennapedia homeobox peptide regulates neural morphogenesis. Proc Natl Acad Sci USA, 1991. 88 (5): p. 1864-8). In 1994, the first 16-mer CPP peptide called penetratin, which has the amino acid sequence RQIKIYFQNRRMKwKk (SEQ ID NO:10) was characterized from the third helix of the Antennapedia homeodomain (Derossi, D., et al., The third helix of the Antennapedia homeodomain translocates through biological membranes. J Biol Chem, 1994. 269 (14): p. 10444-50), followed in 1998 by the identification of the minimal domain of TAT, which has the amino acid sequence YGRKKRRQRRR (SEQ ID NO: 9), necessary for protein transduction (Vives, E., P. Brodin, and B. Lebleu, A truncated HIV-1 Tat protein basic domain rapidly translocates through the plasma membrane and accumulates in the cell nucleus. J Biol Chem, 1997.

In certain embodiments, the CPPs described herein can promote the internalization of a cargo (e.g., a polypeptide) into a tissue, organ, or living cell. Examples of a tissue, organ, or living cell are the liver or liver cells (e.g., hepatocytes), a kidney or kidney cell, a tumor or tumor cell, the CNS or CNS cells (e.g., brain and/or spinal cord), the PNS or PNS cells, a lung or lung cells, the vasculature or vascular cells, the skin or skin cells (e.g., dermis cells and/or follicular cells), the eye or ocular cells (e.g., macula, fovea, cornea, and/or retina), bone, gall bladder, spleen, small intestine, large intestine, stomach, pancreas, appendix, urinary bladder, and an ear or cells of the ear (e.g., cells of the inner ear, middle ear, and/or outer ear), inter alia.

Non-limiting examples of cell penetrating peptides include RKKRWFRRRRPKWKK (SEQ ID NO:8); TAT peptide (YGRKKRRQRRR; SEQ ID NO:9); penetratin (RQIKIWFQNRRMKWKK; SEQ ID NO:10); antennapedia peptide (RQIKIWFQNRRMKWKK; SEQ ID NO:11); transportan (GWTLNSAGYLLGKINLKALAALAKKIL; SEQ ID NO:12); VP22 (RKKRRQRRR; SEQ ID NO:13); MAP (KLALKLALKALKAALKLA; SEQ ID NO:14); CADY (CYGALFLGFLGAAGIAKV; SEQ ID NO:15); Pep-1 (KETWWETWWTEWSQPKKKRKV; SEQ ID NO:16); MPG peptide (GALFLGFLGAAGSTMGAWSQPKKKRKV; SEQ ID NO:17); TP10 (AGYLLGKINLKALAALAKKIL; SEQ ID NO:18); PEP-NP (HAIYPRHVVGGRRR; SEQ ID NO:19); SynBI (RRRQRRKKR; SEQ ID NO:20); TP10-Luc (GLFGALFKAGYLLGKINLKALAALAKKILuc; SEQ ID NO:21); PepFect6 (RQIKIWFQNRRMKWYRWYCKYDK; SEQ ID NO:22); CADY-B2 (CYGALFLGFLGAAGIAKVGGLGC; SEQ ID NO:23); transportan-10 (GWTLNSAGYLLGKINLKALAALAKKIL; SEQ ID NO:24); SynB3 (KKWKMRRNQFWVKVQRG; SEQ ID NO:25); TAT-PTD4 (YGRKKRRQRRRGGGG; SEQ ID NO:26); Pen-2 (RQIKIWFQNRRMKWKKKK; SEQ ID NO:27); MPG-DHFR (GALFLGFLGAAGSTMGAWSQPKKKRKV-HHHHHHH; SEQ ID NO:28); MAP-NLS (KLALKLALKALKAALKLAQSARLTTAVARELKV; SEQ ID NO:29); Pep-3 (KETWWETWWTEWSQPKKKRKVGGGC; SEQ ID NO:30); TP10-VEGF (AGYLLGKINLKALAALAKKILVK; SEQ ID NO:31); PEP-1-EGFP (KETWWETWWTEWSQPKKKRKV-ENLYFQG; SEQ ID NO:32); and TAT-GFP (YGRKKRRQRRRGFPVAT; SEQ ID NO:33); and modified derivatives thereof.

The polypeptides of the present disclosure further encompass CPPs, or analogs thereof, having substantially the same effect as the CPPs described herein. Such CPPs include, but are not limited to, a substitution, addition, or deletion mutant of the CPPs described herein (e.g., in which one or two amino acids of the CPPs (e.g., the CPPs of SEQ ID NOs:8-33) are substituted with another amino acid, are deleted, or are added to the polypeptides). Also encompassed are peptides that are substantially homologous to the polypeptides. A variety of sequence alignment software programs are available in the art to facilitate determination of homology or equivalence of any protein to a protein of the invention.

In certain embodiments, in the CPPs of the polypeptide of the present disclosure, D-amino acids may be used instead of, or in addition to, L-amino acids. While glycine lacks a stereocenter, all other amino acids may be D-amino acids, including D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, and D-Val.

Non-limiting examples of cell penetrating polypeptides (CPPs) contemplated for use in the present invention include CPPs having at least 85%, 86%, 87%, 88%, 89%, 90%, 91%, 92%, 93%, 94%, 95%, 96%, 97%, 98%, 99%, 99.5%, or 100% sequence identity to one or more of SEQ ID NOs:8-33, or any fragment thereof (e.g., fragments of at least 3, 5, 10 or more consecutive amino acids in length), in particular, the sequences of SEQ ID NOs:8-33.

### Methods

In another aspect, the present disclosure provides a method of treating, preventing, and/or ameliorating a disease or disorder in a subject, the method comprising administering to the subject a therapeutically effective amount of a construct comprising a polypeptide comprising amino acid residues 581-635 of soluble guanylyl cyclase (GC1) (SEQ ID NO:34).

In certain embodiments, the amino acid residues 581-635 of GC1 comprise SVFAGVVGGKMPRYCLFGNNVTLANKFESCSVPRKINVSPTTYRLLKDCPGFVFT (SEQ ID NO:7).

In certain embodiments, the polypeptide comprises a fragment of the amino acid residues 581-635 of GC. In certain embodiments, the polypeptide comprises amino acid residues 601-624 of GC1, or a fragment thereof.

In certain embodiments, the fragment comprises 7, 8, 9, 10, 11, 12, 13, 14, 15, 16, 17, 18, 19, or 20 amino acid residues of the amino acid residues 581-635 of GC. In certain embodiments, the fragment comprises 11 amino acid residues.

In certain embodiments, the N-terminus of the fragment corresponds to the amino acid residue 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, or 634 of GC-1. In certain embodiments, the C-terminus of the fragment corresponds to the amino acid residue 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, or 635 of GC-1.

In certain embodiments, the fragment consists essentially of any one of SEQ ID NOs:1-3. In certain embodiments, the fragment consists essentially of SEQ ID NO:3.

In certain embodiments, the polypeptide is conjugated to at least one cell penetrating peptide (CPP). In certain embodiments, the at least one CPP shares 85%, 86%, 87%, 88%, 89%, 90%, 91%, 92%, 93%, 94%, 95%, 96%, 97%, 98%, 99%, 99.5%, or 100% sequence identity with any of SEQ ID NOs:8-33. In certain embodiments, the at least one CPP comprises SEQ ID NO:8.

In certain embodiments, the amino acid residues 581-635 of GC1, or the fragment thereof, are covalently linked to the at least one CPP via an N-terminus amino group of the polypeptide, a C-terminus carboxyl group, and/or a nucleophilic moiety of an amino acid side chain. In certain embodiments, the amino acid residues 581-635 of GC1, or the fragment thereof, are covalently linked to the CPP via the N-terminus amino group of the polypeptide. In certain embodiments, the covalent linkage comprises an amide bond. In certain embodiments, the CPP is conjugated to the polypeptide via a linker. In certain embodiments, the linker is any linker contemplated by one skilled in the art, such as a peptide comprising 1-50 independently selected amino acids, a polyethylene glycol group comprising 1-100 —(CH₂CH₂O)— groups, and/or a C₁-C₁₀₀ alkyl chain, for example.

In certain embodiments, the construct shares 85%, 86%, 87%, 88%, 89%, 90%, 91%, 92%, 93%, 94%, 95%, 96%, 97%, 98%, 99%, 99.5%, or 100% sequence identity with any of SEQ ID NO:5. In certain embodiments, the construct is SEQ ID NO:5.

In certain embodiments, at least one amino acid residue of the polypeptide is derivatized.

In certain embodiments, the derivatization comprises at least one selected from the group consisting of methylation, amidation, and acetylation. In certain embodiments, the derivatization comprises conjugation to a fluorophore. In certain embodiments, the fluorophore is fluorescein isothiocyanate (FITC).

In certain embodiments, the construct is administered as a pharmaceutical composition. In certain embodiments, formation of a GC1/Trx1 complex is inhibited.

In certain embodiments, the disease or disorder comprises cancer.

In certain embodiments, the cancer is of the prostate, lymphatic system, bladder, blood, bone, bone marrow, brain, breast, colon, esophagus, gastrointestine, gum, head, kidney, liver, lung, nasopharynx, neck, ovary, skin, stomach, testis, tongue, and/or uterus.

In addition, the cancer may specifically be at least one of the following histological types, though it is not limited to these: neoplasm, malignant; carcinoma; carcinoma, undifferentiated; giant or spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant-cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast-cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast-cell leukemia; megakaryoblastic leukemia; myeloid sarcoma; and hairy cell leukemia.

In certain embodiments, the reduction in growth of hyperproliferative cells is observed. In certain embodiments, a reduction in viability of hyperproliferative cells is observed.

In certain embodiments, the disease or disorder is a blood pressure disease or disorder.

In certain embodiments, the blood pressure disease or disorder which is selected from the group consisting of hypertension, hypotension, and cardiac dysfunction.

In certain embodiments, the subject is a mammal. In certain embodiments, the mammal is a human.

### Pharmaceutical Compositions

In another aspect, the present disclosure provides a pharmaceutical composition comprising the polypeptide of the present disclosure and at least one pharmaceutically acceptable carrier. The terms “construct of the present disclosure”, “polypeptide of the present disclosure”, and “compound of the present disclosure” may be used interchangeably herein. In certain embodiments, the pharmaceutical composition comprises the polypeptide of the present disclosure, together with one or more pharmaceutically carriers thereof and optionally one or more other therapeutic ingredients. The carrier(s) may be “acceptable” in the sense of being compatible with the other ingredients of the formulation and not deleterious to the recipient thereof.

The pharmaceutical compositions may be manufactured in any manner known in the art, e.g., by means of conventional mixing, dissolving, granulating, dragee-making, levigating, emulsifying, encapsulating, entrapping or compression processes. “pharmaceutically acceptable carrier” may refer to an excipient, carrier or adjuvant that can be administered to a patient, together with at least one therapeutic compound, and which does not destroy the pharmacological activity thereof and is generally safe, nontoxic and neither biologically nor otherwise undesirable when administered in doses sufficient to deliver a therapeutic amount of the compound.

The pharmaceutical formulations may include those suitable for oral, parenteral (including subcutaneous, intradermal, intramuscular, intravenous, intraarticular, and intramedullary), intraperitoneal, transmucosal, transdermal, intranasal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association the polypeptide of the present disclosure (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds of the present disclosure suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, a compound of the present disclosure may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

In certain embodiments, compounds as disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of the present disclosure may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds disclosed herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Intranasal delivery, in particular, may be useful for delivering compounds to the CNS. It had been shown that intranasal drug administration is a noninvasive method of bypassing the blood-brain barrier (BBB) to deliver neurotrophins and other therapeutic agents to the brain and spinal cord. Delivery from the nose to the CNS occurs within minutes along both the olfactory and trigeminal neural pathways. Intranasal delivery occurs by an extracellular route and does not require that drugs bind to any receptor or undergo axonal transport. Intranasal delivery also targets the nasal associated lymphatic tissues (NALT) and deep cervical lymph nodes. In addition, intranasally administered therapeutics are observed at high levels in the blood vessel walls and perivascular spaces of the cerebrovasculature. Using this intranasal method in animal models, researchers have successfully reduced stroke damage, reversed Alzheimer's neurodegeneration, reduced anxiety, improved memory, stimulated cerebral neurogenesis, and treated brain tumors.

In certain embodiments, unit dosage formulations are those containing an effective dose or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

### Combination Therapies

In certain embodiments, the compounds of the present invention are useful in the methods of present invention in combination with one or more additional compounds useful for treating the diseases or disorders contemplated within the invention. These additional compounds may comprise compounds of the present invention or compounds, e.g., commercially available compounds, known to treat, prevent, or reduce the symptoms of the diseases or disorders contemplated within the invention.

Non-limiting examples of additional compounds contemplated within the invention include chemotherapeutic agents, anti-cell proliferation agents, gene therapy agents, immunotherapy agents, and radiation. In certain embodiments, the compounds contemplated within the invention can be used in combination with one or more compounds selected from, but not necessarily limited to, the group consisting of taxotere, cyclophosphamide, paclitaxel, fluorouracil, doxorubicin, cycloheximide, olaparib. and temozolomide. In other embodiments, the compounds contemplated within the invention can be used in combination with any chemotherapeutic, gene therapy or immunotherapy compound or treatment regimen known in the art. In yet other embodiments, the compounds contemplated within the invention can be used in combination with chemotherapeutic compounds known to treat cancer and/or radiation therapy.

The compounds contemplated within the invention may be administered before, during, after, or throughout administration of any therapeutic agents used in the treatment of a subject's disease or disorder.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions useful within the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the present invention is from about 1 and 5,000 mg/kg of body weight/per day. One of ordinary skill in the art is able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the present invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the present invention are dictated by and directly dependent on the unique characteristics of the therapeutic compound and the particular therapeutic effect to be achieved, and the limitations inherent in the art of compounding/formulating such a therapeutic compound for the treatment of a disease or disorder in a patient.

In certain embodiments, the compositions useful within the invention are formulated using one or more pharmaceutically acceptable excipients or carriers. In certain embodiments, the pharmaceutical compositions of the present invention comprise a therapeutically effective amount of a compound useful within the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate, or gelatin.

In certain embodiments, the compositions useful within the invention are administered to the patient in dosages that range from one to five times per day or more. In other embodiments, the compositions useful within the invention are administered to the patient in range of dosages that include, but are not limited to, once every day, every two days, every three days to once a week, and once every two weeks. It is readily apparent to one skilled in the art that the frequency of administration of the various combination compositions useful within the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physician taking all other factors about the patient into account.

Compounds for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound is from about 1 mg and about 2,500 mg. In some embodiments, a dose of a compound of the present invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating a disease or disorder) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the drug is therapeutically active at a circulating and/or tissue concentration of about 1, 2, 4, 6, 8, 10, 12, 14, 16, 18, 20, 25, 30, 35, 40, 45 or 50 μM.

In certain embodiments, the present invention is directed to a packaged pharmaceutical composition comprising a container holding a therapeutically effective amount of a compound of the present invention, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like. They may also be combined where desired with other active agents, e.g., other anti-tumor agents.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

Routes of administration of any of the compositions of the present invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, particularly suitable are tablets, dragees, liquids, drops, or capsules, caplets and gelcaps. The compositions intended for oral use may be prepared according to any method known in the art and such compositions may contain one or more agents selected from the group consisting of inert, non-toxic pharmaceutically acceptable excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the present invention may be administered in the form of microparticles, for example, by injection or in the form of wafers or discs by implantation.

In certain embodiments, the compounds of the present invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound depends on the age, sex and weight of the patient, the current medical condition of the patient and the progression of the disease or disorder in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the present invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions with art-recognized alternatives and using no more than routine experimentation, are within the scope of the present application.

It should be understood that the method and compositions that would be useful in the present invention are not limited to the particular formulations set forth in the examples. The following examples are put forth so as to provide those of ordinary skill in the art with a complete disclosure and description of how to make and use the composition and therapeutic methods of the invention, and are not intended to limit the scope of what the inventor regard as his invention.

The following examples further illustrate aspects of the present invention. However, they are in no way a limitation of the teachings or disclosure of the present invention as set forth herein.

### EXAMPLES

Various embodiments of the present application can be better understood by reference to the following Examples which are offered by way of illustration. The scope of the present application is not limited to the Examples given herein.

### Materials and Methods

### Reagents

Purified recombinant human GC1 protein was purchased from Enzo life sciences (ALX-201-177-C010), Thioredoxin 1 (Trx1) protein was from Fitzgerald industries (30R-2985). FBS was from VWR (97068-085), GC1 α antibody was from ThermoFisher (MA517086), GC1 β antibody from Cayman chemical company (160897), Trx1 antibodies were from Cell signaling (2298S and human specific: 2285S). Caspase 3 antibody was purchased from Novus biologicals (NB100-56708) and biotin antibody from Abcam (ab1227). The following reagents were purchased from Sigma: DTT (D0632), diamide (D3648), Amicon Ultra-0.5 centrifugal filter (UFC500324), Bradford reagent (B6916), PMSF (P7626), protease inhibitor cocktail (P8340), Etoposide (E1383) and the Caspase 3 activity assay kit (CASP3C-1KT). [α-³²P]-GTP was from PerkinElmer (NEG006H250UC), S-nitrosoglutathione from Calbiochem (487920). jetPRIME transfection kit was from Polyplus transfection (101000015), Diethylamine NONOate (DEA-NO) from Enzo life sciences (ALX-430-034-5005) and biotin-HPDP from ThermoFisher (21341). Jurkat T cell line, clone E6-1 was purchased from ATCC (TIB-152).

### Peptides Synthesis

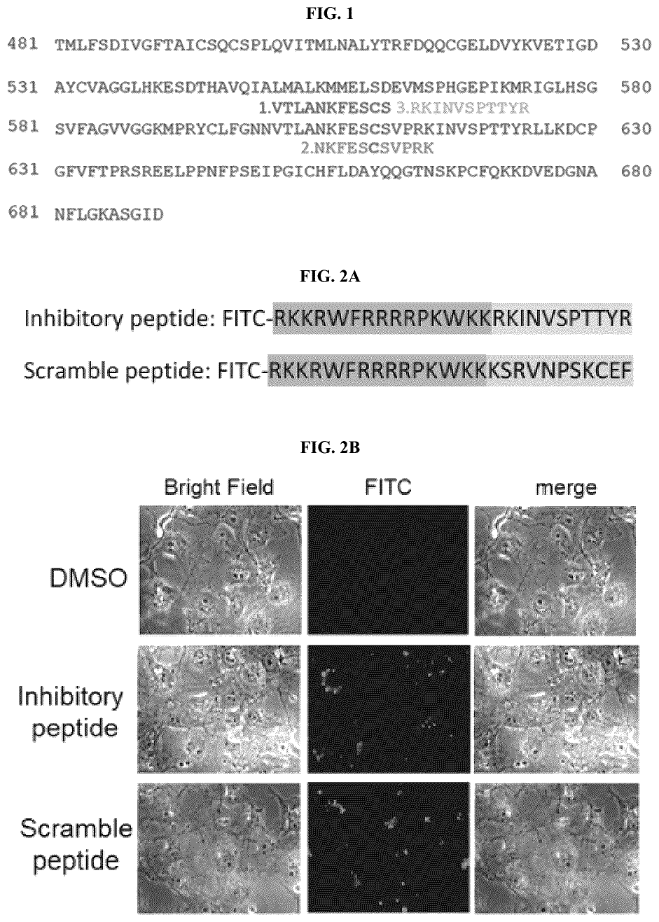

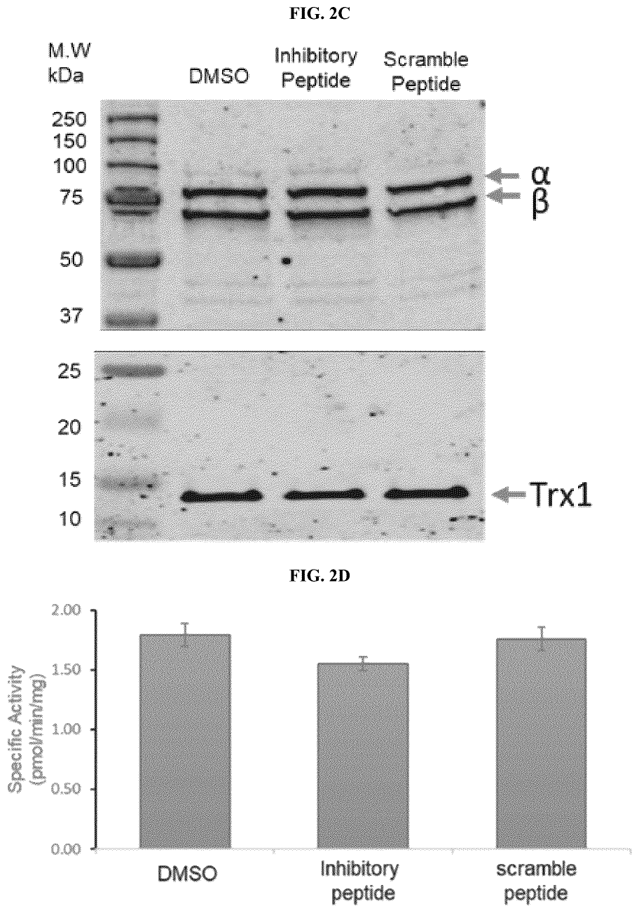

Two sets of peptides were synthesized. The first set of three 11-amino acid peptides, synthesized by Epoch Life science, mimic the sequence of amino acids in proximity of, or overlapping, Cysteine 610 of the α subunit of GC1. A fourth scramble peptide was synthesized as control. The sequences are provided in FIG. 1. As the 3 peptides were efficient at disrupting the GC1/Trx1 complex in vitro, peptide 3 (inhibitory peptide) and the scramble peptide were modified (Abclonal Science) for cell penetration by addition of a tat/penetrating hybrid peptide and FITC (FIGS. 2A-2D)

### Cell Culture and Transfection

COS-7 cells (ATCC) were cultured in a 6-well plate with a complete medium DMEM from Corning (10-013-CV) supplemented with 10% FBS (heat inactivated) and Pen-Strep (ThermoFisher, 15140122) at 37° C. with 5% CO₂. The cells were transfected with the wild-type GC1 α and β subunits from rat, subcloned in pCMV5 vector using the jetPRIME kit. After 4 h, the medium was replaced with complete medium supplemented with 10% FBS+P/S. After 24 h transfection, the cells were washed twice and cultured in serum-free medium for peptide treatment.

Jurkat cells were cultured in a 6-well plate at the density of 0.25×10⁶/ml in complete medium RPMI 1640 from Corning (10-040-CV) supplemented with 10% FBS and Pen-Strep and incubated at 37° C. with 5% CO₂ for 24 h. The cells were collected by centrifuging at 1000 g for 5 min, washed twice with serum-free medium, and re-suspended with 2 ml serum-free medium prior to peptide treatment.

### Peptides Treatment and Assay of GC1 Activity in a Purified System

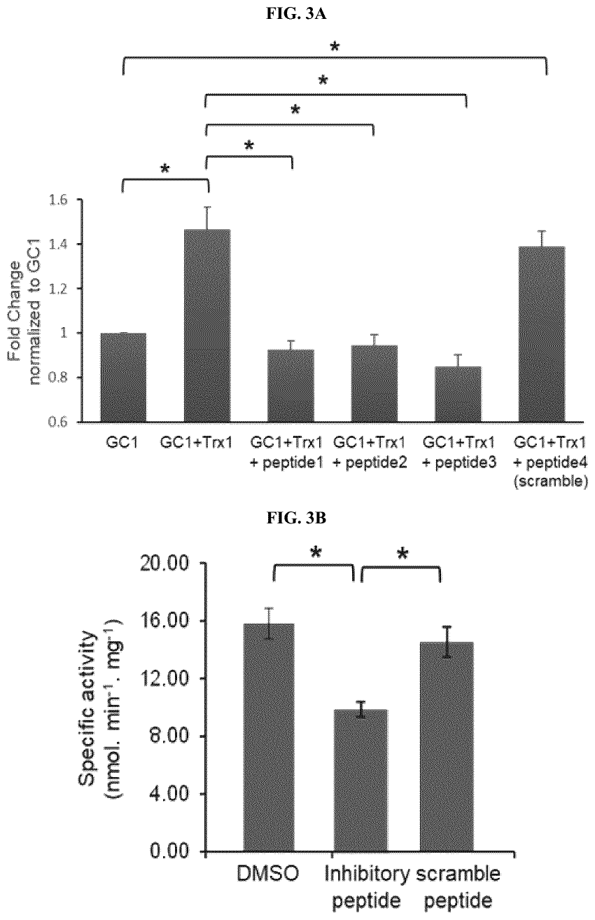

Each peptide from set 1 (FIG. 1) was dissolved in 70% DMSO to obtain a 10 mM stock solution and further diluted with H₂O to have a 250 μM working stock. In each reaction tube, 0.25 μg of human recombinant GC1 was mixed with 0.15 μg of Trx1 (molar ratio GC1:Trx1˜1:7) or not (to control the effect of peptides on GC1 activity). In each tube of the GC1 and Trx1 mixture or GC1 alone, 5 μL of each peptide were added in a 50 μL final volume of 50 mM HEPES, pH 8.0. The final concentration of each peptide was 25 μM. The molar ratio of GC1 to each peptide was 1:800. The tubes were incubated on ice for 20 min in the dark. After incubation, the samples were subjected to GC1 activity assay under basal and NO-stimulated conditions. The GC1 activity was measured by the conversion of [α-³²P]-cGMP from [α-³²P]-GTP, as previously described. Fifty L reaction mix containing 50 mM HEPES, pH 8.0, 5 mM MgCl₂, and 0.5 mM GTP were added to each sample and incubated at 30° C. for 5 min. DEA-NO (1 μM final concentration) was used as the NO donor to stimulate GC1 activity. The reaction was stopped by adding 500 μL of 120 mM zinc acetate, followed by addition of 500 μL of 144 mM sodium carbonate. [α-³²P]-cGMP was separated using alumina columns. The recovery rate was calculated using 100 μL of cGMP-³H. The specific activity was first expressed in nmol cGMP·min⁻¹·mg⁻¹ (FIG. 11 and FIG. 3B), the values from FIG. 11 were then expressed as a fold change normalized to GC1 NO-stimulated activity alone (FIG. 3A); n=4 independent experiments in duplicate.

### Peptides Treatment and Assay of GC1 Activity in COS-7 Cells.

After 24 h transfection with WT GC1, COS-7 cells were either treated with 70% DMSO (0.35% final), the selected inhibitory peptide, or scramble peptide at 37° C. for 1 h in serum-free medium. The final concentration of each peptide was 5-10 μM (sequences are provided in FIG. 2A). After 1 h incubation, the cells were washed twice and incubated in the complete medium at 37° C. for 24 h. After 24 h incubation, the cells were washed 3 times with ice-cold PBS and sonicated in homogenization buffer containing 50 mM HEPES, pH 8.0 and 150 mM NaCl supplemented with PMSF and protease inhibitor cocktail. The protein concentration of each sample was measured using the Bradford method. 20 μg protein of each sample was used for GC1 activity assay under both basal and NO-stimulated conditions. The GC1 activity assay was conducted as above. Prior to lysis, cover slips were collected at 12 h and 24 h to visualize FITC fluorescence and assess peptides penetration (FIG. 2B). Conversely, expression of GC1 α and β subunits in COS-7 cells and endogenous expression of Trx1 were assayed by Western blot (FIG. 2C).

### Peptide Treatments and Assays of Reduction of Oxidized GC1 by Reduced Trx1 and of Oxidized Trx1 by Reduced GC1, in a Purified System

1 μg of recombinant human GC1 was treated with 10 mM DTT at 37° C. for 30 min in the dark or with 100 μM diamide on ice for 30 min in the dark to generate reduced (rGC1) and oxidized GC1 (oGC1), respectively. Four hundred ng of recombinant human Trx1 was treated with 10 mM DTT at 37° C. for 30 min in the dark or with 100 μM diamide on ice for 30 min in the dark to generate the reduced (rTrx1) and oxidized Trx1 (oTrx1), respectively. The proteins were transferred onto the Amicon Ultra-0.5 centrifugal filters, centrifuged at 14000 g for 10 min, and washed 3 times with a buffer containing 50 mM HEPES, pH 8.0 and 5 mM EDTA to remove excess reagent. The proteins were recovered by flipping the filters and centrifuging at 1000 g for 2 min. Various combinations of reduced or oxidized forms of GC1 and Trx1 with or without peptides were mixed and incubated at room temperature for 30 min in the dark. In each tube, GC1:Trx1 molar ratio was estimated to be 1:5; the concentration of peptides, if added, was 125 μM final. The samples were analyzed by Western blots: gel (12% SDS-PAGE) electrophoresis was conducted under reducing and non-reducing conditions, transferred on nitrocellulose membrane and then probed for either Trx1 or GC1.

### Peptides Treatment of GC1-Trx1 Transnitrosation Reaction in a Purified System

Recombinant human GC1 was treated with 100 μM S-nitrosoglutathione (GSNO) at 37° C. for 30 min in the dark to generate SNO-GC1, as previously described. Recombinant human Trx1 was treated with 10 mM DTT at 37° C. for 30 min in the dark or with 100 μM diamide on ice for 30 min in the dark to generate the rTrx1 and oTrx1, respectively and the excess reagents removed as above. oTrx1 was treated with 100 μM GSNO at 37° C. for 30 min in the dark to generate SNO-oTrx1, as previously described. The proteins were transferred onto Amicon Ultra-0.5 centrifugal filters, centrifuged at 14000 g for 10 min, and washed 3 times with a buffer containing 50 mM HEPES, pH 8.0 and 5 mM EDTA to remove excess reagent. The proteins were recovered by flipping the filters and centrifuging at 1000 g for 2 min. Various combinations of SNO-GC1, oTrx1, or rTrx1 with or without peptides were mixed (GC1:Trx1 molar ratio was estimated at 1:5, the final concentration of each peptide was 125 μM) and incubated at room temperature for 30 min in the dark. The samples were analyzed by biotin or biotin/avidin switch assays as previously described, followed by Western blots. To detect biotinylated proteins, the samples were electrophorated under non-reducing conditions and probe with anti-biotin antibody (1:3000, Abcam), the inputs (starting material) were electrophorated under reducing conditions and probe for Trx1 and GC1.

### Biotin Switch Assay and Avidin Enrichment

Free thiols of proteins were blocked in lysis-blocking buffer containing 50 mM Tris, pH 7.5, 150 mM NaCl, 1% Triton X-100, 1 mM EDTA, 2% SDS, 0.1 mM neocuproine, 0.2 mM PSMF, and 40 mM N-ethylmaleimide (NEM) at 50° C. for 30 min in the dark. Excess NEM was removed using cold acetone precipitation. The protein pellets were generated by centrifugating at 14000 g for 10 min at 4° C. and washed 3 times using cold acetone. The pellets were resuspended in 300 μL of a buffer containing 25 mM HEPES, pH 7.7, 1 mM EDTA, and 1% SDS, supplemented with 0.8 mM biotin-HPDP and 10 mM ascorbate, at room temperature for 1 h in the dark. Negative controls were the blocked samples without ascorbate. Excess biotin-HPDP was removed and biotinylated proteins were precipitated using cold acetone at −20° C. for 1 h followed by centrifugation at 5000 g for 10 min at 4° C. The pellets were dissolved in HENS buffer containing 50 mM Tris pH 7.5, 150 mM NaCl, 1% Triton X-100, and 0.5% SDS. For detection of biotinylated proteins, resuspended samples were mixed with 4× Laemmli buffer without β-Mercaptoethanol.

For avidin enrichment, the biotinylated samples were diluted in 700 μL PBS and mixed with 100 μL streptavidin-agarose beads. The mixture was incubated for 1 h at room temperature with regular agitation. The beads were pelleted by centrifuging at 5000 g for 10 min and washed 3 times with 1 ml of phosphate buffered saline, mixed with 100 μL of 1× Laemmli loading buffer with 10% β-Mercaptoethanol and heated at 85° C. for 5 min. The proteins released from the beads were then subjected to Western blotting.

Jurkat T Cells Treatment with Peptides and Etoposide-Induced Apoptosis

The cells in suspension in 2 ml serum-free medium were treated with either 70% DMSO (20 μL, 0.7% final), scramble peptide, or inhibitory peptide at a final concentration of 10 μM and incubated at 37° C. for 1 h. After incubation, the cells were washed twice and incubated in complete medium at 37° C. for 8 h. The cells were then treated with (or without) etoposide (ETO) at 8 μM final at 37° C. for 16 h.

For biotin-avidin switch assays: After 16 h incubation, the cells were washed twice with cold PBS and lysed in lysis-blocking buffer containing 50 mM Tris, pH 7.5, 150 mM NaCl, 1% Triton X-100, 1 mM EDTA, 2% SDS, 0.1 mM neocuproine, and 40 mM NEM supplemented with PMSF and protease inhibitor cocktail. The protein concentration was measured using the Bradford method. 50 μg of each lysate was analyzed by Western blot as starting material (inputs) and 200 μg proteins of each sample were subjected to the biotin switch and avidin assays, as described elsewhere herein. Blots under non-reducing conditions were probed with anti-biotin. Under reducing conditions following avidin assays, Blots were probed with anti-GC1 α and β (1:1000), anti-Caspase 3 (1:500) and anti-Trx1 (1:500).

For Caspase-3 activity assays: After 16 h incubation, the cells were washed twice with cold PBS and lysed in the supplier's 1× lysis buffer (at 10 μL per 10⁶ cells). After 15 min incubation on ice, the lysates were centrifuged at 16,000 g for 15 min at 4° C. and the supernatant collected. Caspase-3 activity was measured following the supplier's instructions (CASP3C-1KT, Sigma) after 2 and 3 h incubation.

### Statistical Analysis

Value outputs are expressed as average±SEM with n>3. The comparison studies with GC1 NO-stimulated activity were done with a two-tailed student's t-test with a level of 0.05 in the purified system, and using one-way ANOVA followed by Tuckey's posthoc test in the cell studies. Caspase-3 activity was measured in three independent experiments (n=3) and each measurement in duplicate. The means and standard deviation of each measurement was calculated, and the variance was compared using one way ANOVA followed by Sidak's multiple comparison task with p<0.05 using GraphPad Prism v 9.4.1. P<0.05 was considered statistically significant.

### Example 1: Peptides Predicted to Disrupt the Interaction Between GC1 and Trx1 Blunt the Ability of Trx1 to Enhance NO-Stimulated Activity of GC1

It has been previously demonstrated that C610 of the α subunit of GC1 (αC610) is key to the interaction between GC1 and Trx1 under reducing and oxidizing conditions. To gain insight into the function of the two NO signaling pathways initiated by either the reduced or the oxidized GC1/Trx1 complex (i.e., enhanced NO-cGMP forming activity or transnitrosation activity) three peptides were designed with overlapping amino sequence containing the αC610 to disrupt the GC1/Trx1 interaction. The peptides sequences of these constructs are disclosed herein (FIG. 1 and FIGS. 2A-2D).

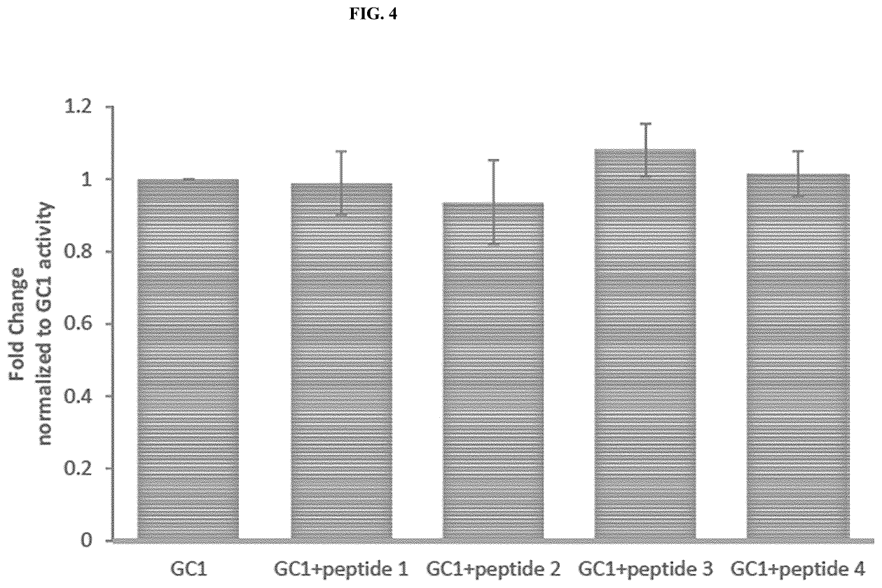

Three peptides and a scramble peptide were first screened as control and assayed their ability to block the Trx1-enhancing effect on GC1 activity. The in vitro system consisted of mixing purified Trx1 and GC1 under reducing conditions. GC1 and Trx1 were mixed at molar ratio of 1:7 while peptides were added to GC1/Trx1 at a molar ratio of GC1:peptides of 1:800. Similarly, GC1 alone was mixed with the 4 different peptides in the absence of Trx1 to control for a direct effect of the peptides on GC1. The different combinations were kept on ice and in the dark for 20 min, then GC1 activity was assayed under basal and NO stimulated conditions (1 μM DEA-NO). FIG. 3A first confirmed the increased NO-stimulated activity of GC1 by Trx1 and showed that Trx1-dependent increase of NO-stimulated GC1 activity was similarly blunted by the 3 peptides while the scramble peptide had no effect. Importantly, none of the peptides affected the NO-stimulated activity in the absence of Trx1 (FIG. 4), suggesting that the peptides do not affect the structure of the catalytically active conformation of GC1, but rather affect the functional interaction between GC1 and Trx1. Under basal conditions, Trx1 did not stimulate GC1 activity, as previously observed in cells and conversely the peptides had no effect.

Whether these peptides could affect Trx1 enhancement of GC1 activity in a cellular context was next assessed. One inhibitory peptide (i.e., peptide 3) was selected, and modified, together with the scramble peptide, to make it cell permeable and fluorescent (see sequence in FIG. 2A).

Successful addition of the peptide in the cytosol was confirmed by fluorescent imaging (FIG. 2B). COS-7 cells were used, which do not express GC1 but have detectable level of Trx1 and transfected them with GC1 expressing vector (Western blot, FIG. 2C), and 10 μM of inhibitory or scramble peptides or DMSO were added. Cell lysates were then assayed for GC1 activity. As shown in FIG. 3B, addition of the inhibitory peptide significantly decreased NO-stimulated GC1 activity compared to the control (DMSO) and the scramble peptide. The peptides had no effect on basal activity (FIG. 2D).

Thus, this inhibitory peptide in vitro and in cells interferes with the GC1/Trx1 interaction, in turn blunting the enhancing effect of Trx1 on NO-stimulated GC1 activity.

### Example 2: Bidirectional Reduction of oGC1 by rTrx1 and oTrx1 by rGC1 is Disrupted by the Interface-Inhibitory Peptide

It has been previously hypothesized that Trx1 was enhancing NO stimulated activity by reducing thiol oxidation of GC1. This hypothesis was evaluated by determining whether reduced Trx1 (rTrx1) could reduce oxidized GC1 (oGC1) and if this effect was abrogated by the inhibitory peptide.

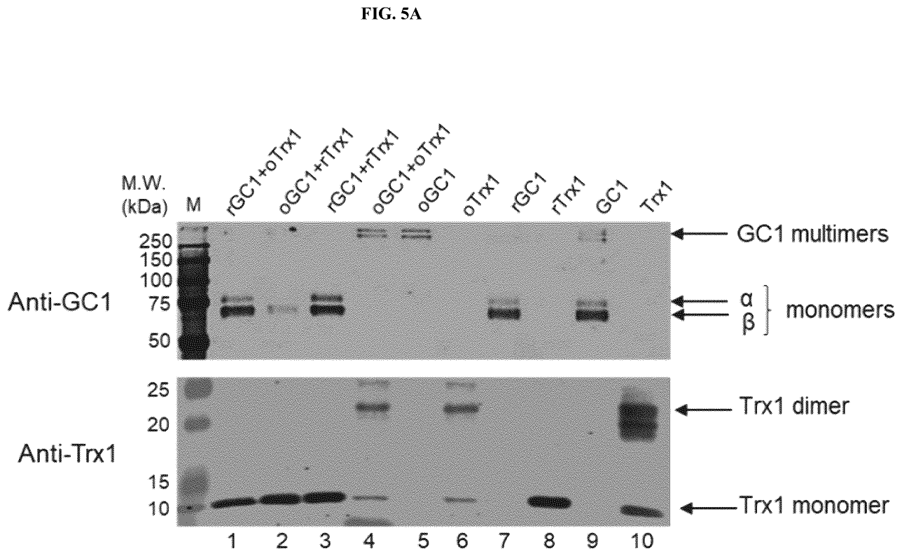

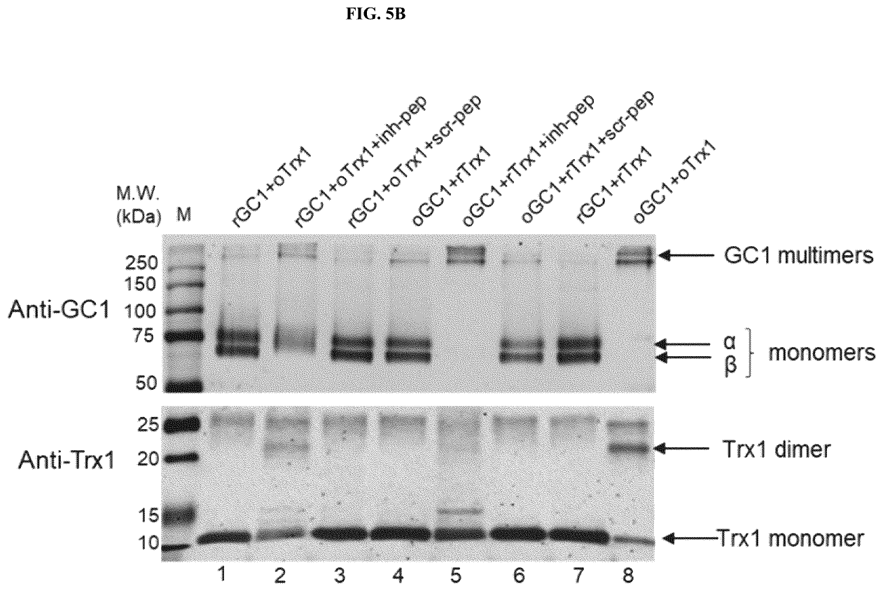

Using non-reducing electrophoresis, the ability of rTrx1 to reduce oGC1 was first assayed. As a control, the effect of reduced GC1 (rGC1) on oTrx1 was also assayed. GC1 and Trx1 were both oxidized using 100 μM diamide or reduced using 10 mM DTT. After removing excess of reductants and oxidants, the different combinations were mixed (molar ratio GC1:Trx1 was 1:5) and incubated in the dark at room temperature for 30 min then subjected to non-reducing and reducing electrophoreses. FIG. 5A of non-reducing gel probed for GC1 and Trx1 indicated that oxidation of GC1 induces conversion of the α and β monomers of reduced GC1 into high molecular weight multimers (compared lane 7 with lane 5, upper panel FIG. 5A), while oxidation of Trx1 leads to conversion of the majority of its reduced monomers into dimers (compared lane 8 to lane 6, lower panel FIG. 5A), as previously observed. Lanes 9 and 10 are untreated GC1 and Trx1, respectively, both showing a mixed monomers/multimers forms.

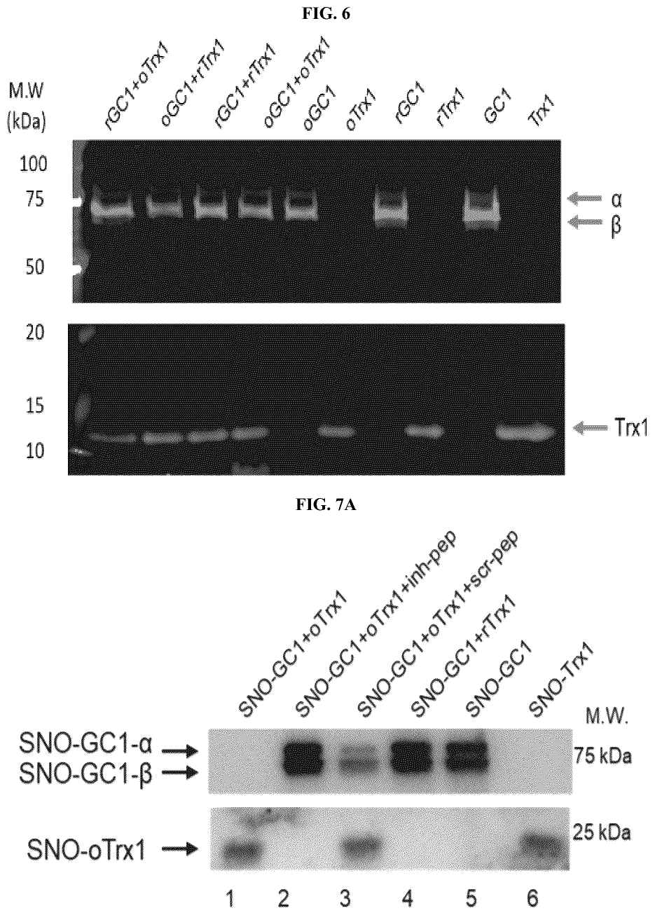

rTrx1 converted oGC1 multimers into monomers to some extent (lane 5 vs. lane 2, upper panel; though it is better seen in FIG. 5B, upper panel lane 4) and unexpectedly, rGC1 converted most of oTrx1 dimer into Trx1 monomers (lane 6 vs. lane 1 lower panel of FIG. 5A). The reducing gel of the same reactions is provided in FIG. 6. The ability of each rGC1 and rTrx1 to reduce their oxidized counterparts of the complex confirmed that these reactions are driven by the redox state of both molecules. In addition, the observation that rGC1 reduces oTrx1, hence potentially restores Trx1 reductase activity is novel and suggests that GC1 is a regulator of the thiol-redox state of cells via reduction of inactive oTrx1.

Using the same conditions, whether the inhibitory peptide could suppress the reductase activity of GC1 toward oTrx1, and vice-versa, was evaluated. The oxidation and reduction of GC1 and Trx1 were conducted as above and the different mixes incubated with 125 μM of the inhibitory peptide (Inh-pep, the original peptide 3 of FIG. 3A) or scramble peptide (Scr-pep) for 30 min at room temperature in the dark, or DMSO as control. FIG. 5B confirmed that rGC1 has the ability to convert the oTrx1 dimers into monomers (lane 8 vs. lane 1, anti-Trx1 lower panel) but this effect was mostly blunted by the presence of the inhibitory peptide (lane 2 vs. lane 1, lower panel).

As a control, the rGC1-dependent conversation of Trx1 from dimers to monomers was maintained in the presence of the scramble peptide (lane 3 compared to lane 8, lower panel). Conversely, rTrx1 is efficient in converting the oGC1 multimers into monomers (lane 4 vs lane 8, upper panel anti-GC1) and this effect was blocked by the inhibitory peptide (compared lane 4 to lane 5, upper panel), and the reducing effect of Trx1 was maintained with the scramble peptide as control (lane 6, upper panel). The ability of the inhibitory peptide to block the reducing reactions between Trx1 and GC1 not only confirmed the reducing potential of rTrx1 and rGC1 toward oGC1 and oTrx1, respectively, but also indicated that this inhibitory peptide is a powerful tool to study the physiological relevance of the complex.

### Example 3: Unidirectional Transnitrosation from SNO-GC1 to oTrx1 is Blunted by the Interface-Inhibitory Peptide

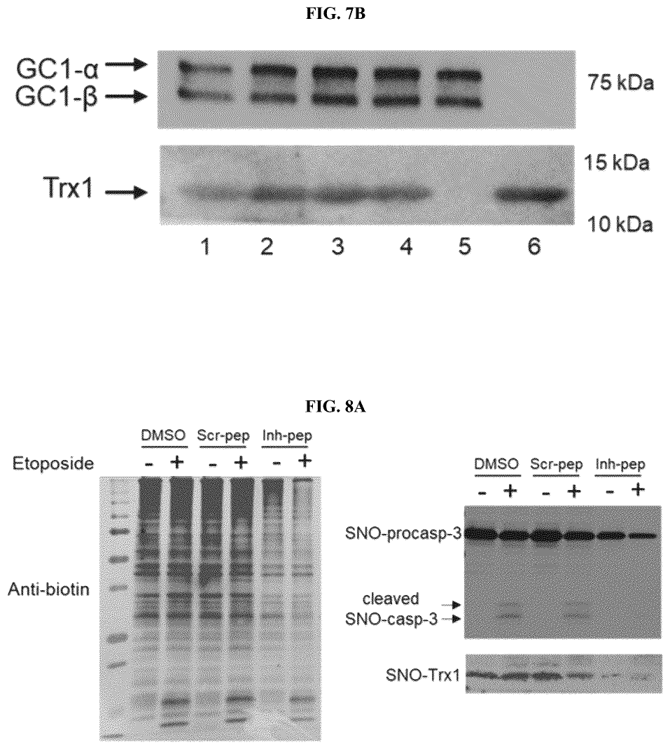

The other important function of this complex is to initiate transnitrosation cascades with SNO-GC1 using oTrx1 as a nitrosothiol relay, under oxidative conditions. It was first determined whether the inhibitory peptide could block this transnitrosation reaction, as it involves the transfer of the nitrosothiols group from αC610 of GC1 to C73 of Trx1. It has been previously demonstrated that only the oxidized form of Trx1 (oTrx1) is S-nitrosated, thus Trx1 was first oxidized, then oTrx1 or GC1 were treated with GSNO to generate SNO-oTrx1 and SNO-GC1; the latter was mixed with oTrx1. The inhibitory peptide or scramble peptide were added at 125 M to the SNO-GC1+oTrx1 mix (the molar ratio GC1:Trx1 was 1:5). The control was the solvent DMSO at 0.9%. The transfer of the SNO groups between the molecules was assayed by biotin switch assay under non-reducing conditions. FIG. 7A confirmed that SNO-GC1 transnitrosates oTrx1 (lane 1, lower panel) while SNO-GC1 becomes undetectable (compared lane 1 to lane 5, upper panel) indicating a near complete transfer of the SNO from SNO-GC1 to oTrx1. In contrast, this same transnitrosation reaction was completely blocked by the inhibitory peptide as no SNO-oTrx1 (lower panel, lane 2) and no decrease in SNO-GC1 (upper panel, lane 2) were observed. In contrast, the scramble peptide could not inhibit the transnitrosation reaction between SNO-GC1 and oTrx1, as SNO-oTrx1 was still detected (lane 3 lower panel) while the intensity of SNO-GC1 was drastically reduced (lane 3, upper panel). Of note, SNO-GC1 transnitrosation did not occur with rTrx1 (lane 4), as previously observed, confirming that the thiol-redox state is essential in these reactions. FIG. 7B is a Western blot of the same samples electrophorated under reducing conditions.

### Example 4: The Inhibitory Peptide Increases Significantly Caspase-3 Activity in Response to Etoposide while Decreasing S-Nitrosation of Both Caspase-3 and Trx1 in Jurkat T Cells

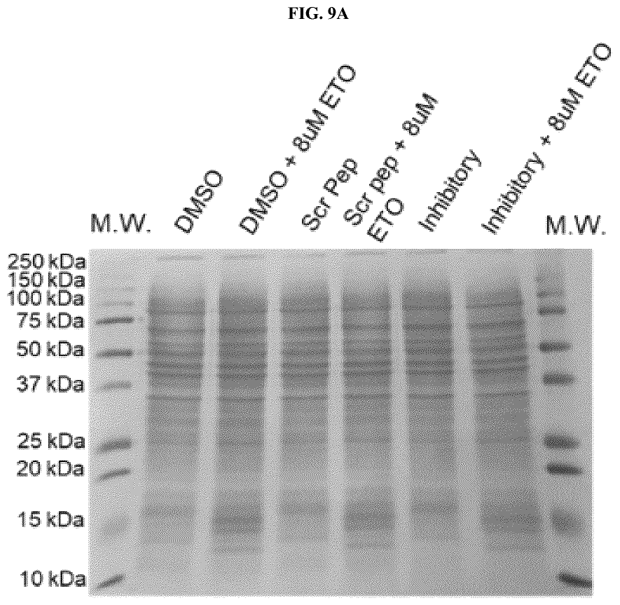

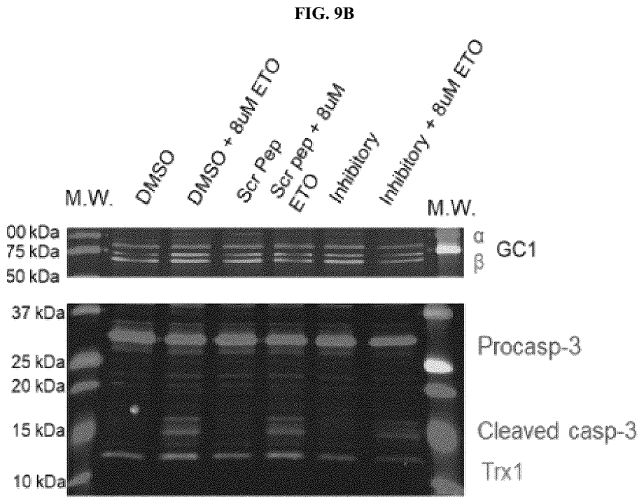

The above experiments were done in a purified system, thus it was sought to determine whether disruption of the GC1-Trx1 transnitrosation complex could have biological relevance in a specific cell type. The proliferative Jurkat T cells (human leukemic T cell line) were used because it was shown that S-nitrosation of procaspase-3 by oTrx1 partially blocked the cleavage into active caspase-3, hence apoptosis. The source of SNO-Trx1 in these cells was unknown and mutational analysis identified C73 as the SNO donor of oTrx1 (to the acceptor C163 of procaspase-3). Since GC1 is expressed in the Jurkat T cells (FIG. 9B), the idea that SNO-GC1 is a source of SNO-Trx1 was evaluated. If so, the GC1/Trx1 complex inhibitory peptide should disrupt the transfer of S-nitrosothiols from GC1 to Trx1, hence the Trx1-dependent S-nitrosation of caspase-3. In turn, an increase in caspase-3 activity and apoptosis in cells treated with the inhibitory peptide is expected.

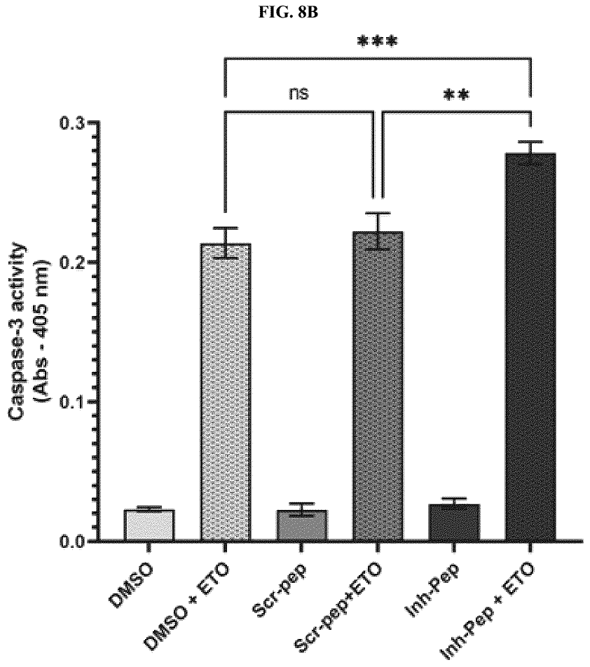

Jurkat T cells were untreated (control, DMSO), treated with the scramble peptide (10 μM, 1 h) or with the inhibitory peptide (10 μM, 1 h) and assayed under basal and apoptotic conditions (etoposide, 8 μM for 16 h). Biotin-avidin assays were first conducted to determine the levels of S-nitrosation under the various conditions. As shown in FIG. 8A, the levels of S-nitrosation are high at baseline in the cells treated with DMSO and scramble peptide but there was a visible decrease in global SNO in the cells treated with the inhibitory peptide. Etoposide (ETO) did not affect the overall level of S-nitrosation (left panel). The biotinylated samples were then avidin purified, and after electrophoresis, the blots were probed with caspase-3 and Trx1 antibodies (right panel, FIG. 8A). SNO-procaspase-3 was greatly reduced in the cells treated with the peptide inhibitor, compared to DMSO and scramble peptide treated cells; S-nitrosation of caspase-3 cleaved fragments was detectable with ETO treatment in the controls (DMSO, scramble peptide) but no SNO-caspase-3 cleaved fragments could be detected in cells with the inhibitory peptide.

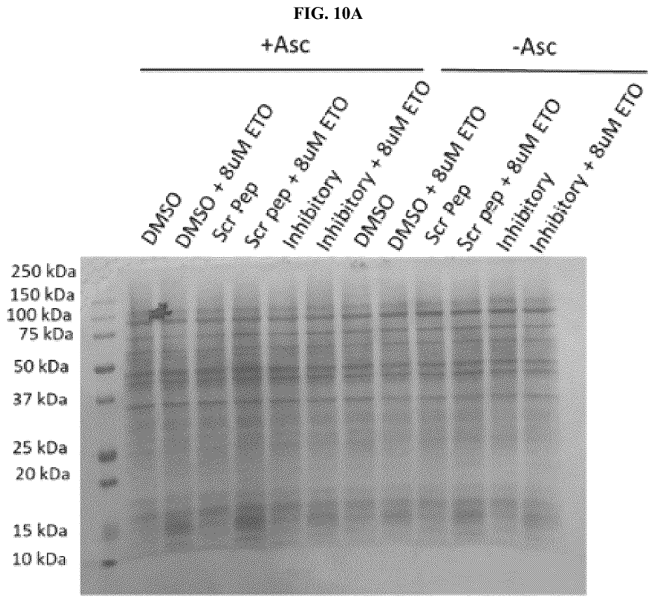

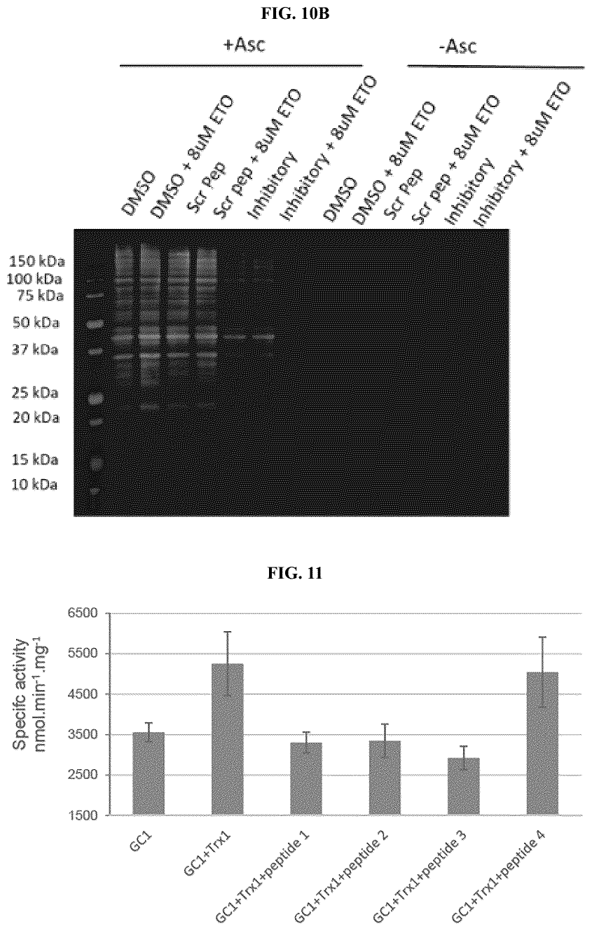

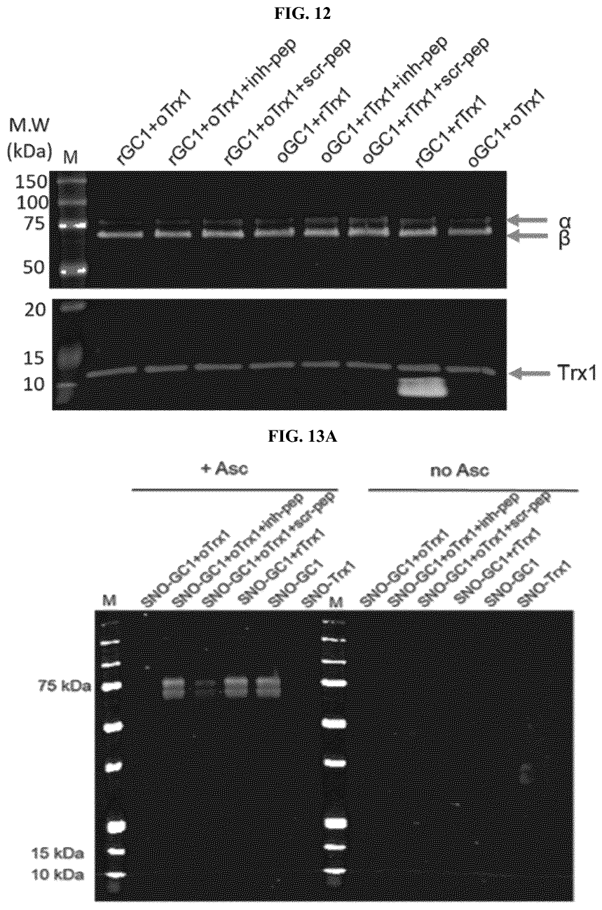

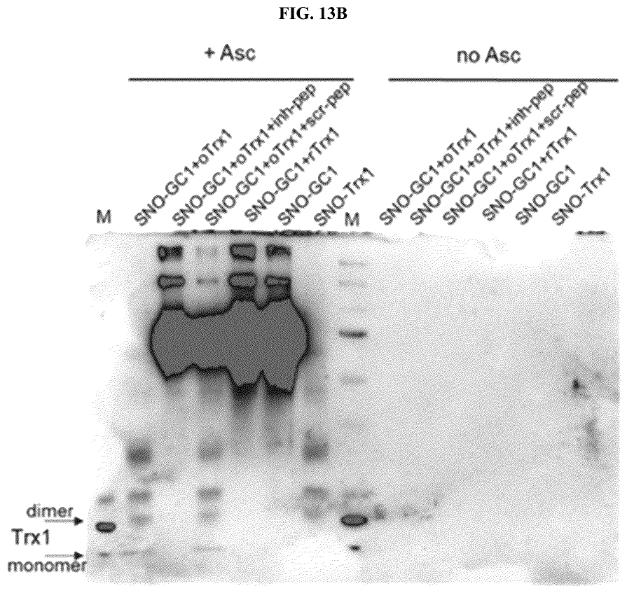

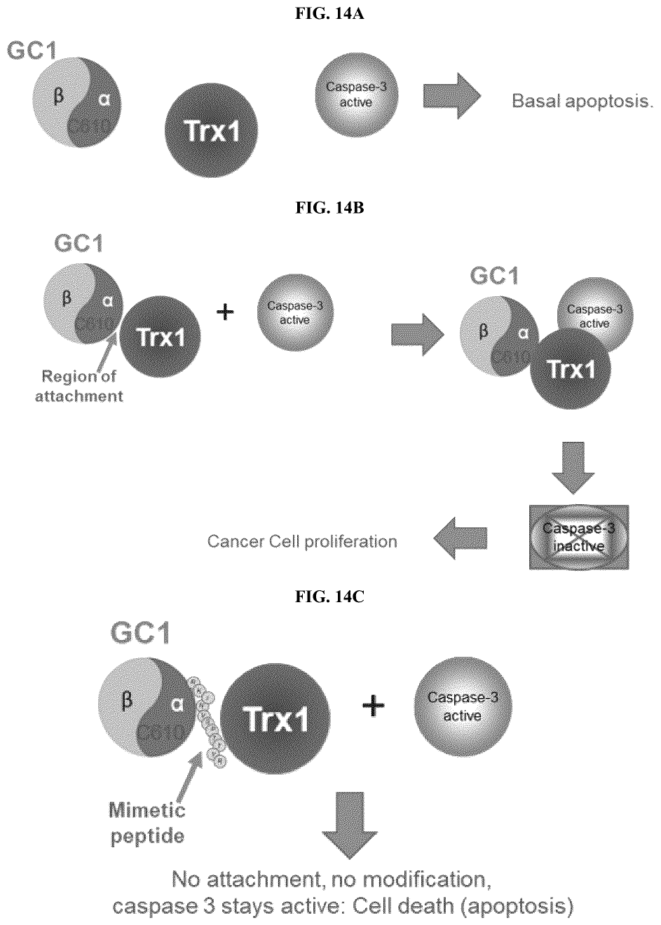

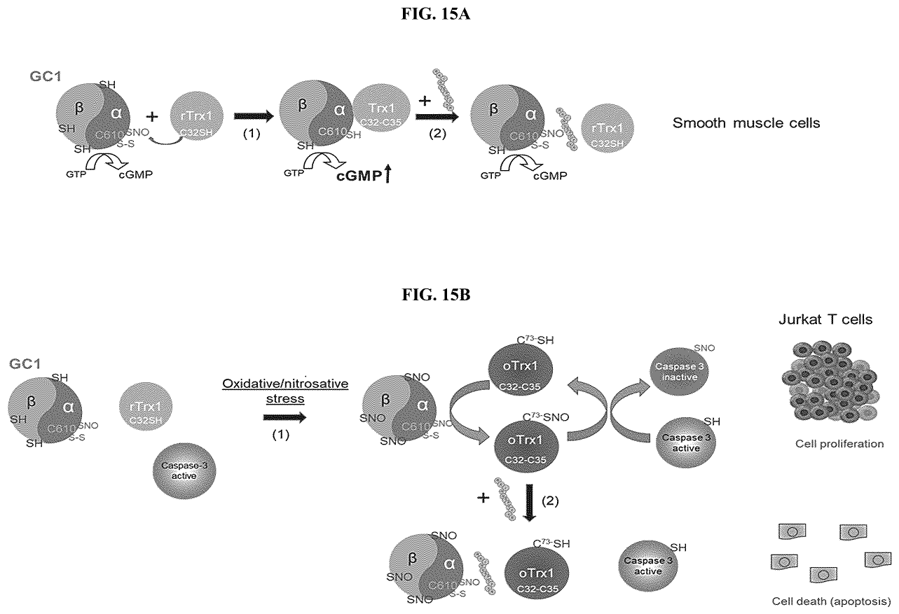

Conversely, inhibitory peptide strongly decreased the levels of SNO-Trx1, compared to the controls (DMSO and scramble peptide) confirming, in cells, the ability of the inhibitory peptide to block the SNO-GC1 to Trx1 transnitrosation reaction. The corresponding stained Ponceau red and uncropped blots that include controls without ascorbate are provided in FIGS. 10A-10B. Of note, Western blot of the inputs (cell lysates of the starting material) of FIG. 9B, showed some reduced procaspase-3 levels of the cells treated with the inhibitory peptides in response to the ETO treatment, compared to the controls treated with ETO. Ponceau red staining of the same blot showed a similar amount of proteins in each sample (FIG. 9A).

Together, this suggested that the lower level of S-nitrosated procaspase-3 resulted in its increased processing in cells where the GC1/Trx1 complex is disrupted. Whether these observations correlated with an increased caspase-3 activity was next assessed with a colorimetric assay based on cleavage of the substrate DVED, under the same above conditions. As shown in FIG. 8B, treatment with ETO induced a significant increase in caspase-3 activity compared to samples not treated with ETO, as expected. More importantly, in the Jurkat cells treated with the inhibitory peptide, the apoptotic activity was strongly and significantly increased compared to the cells treated with DMSO or scramble peptide. It was concluded that disrupting the SNO-GC1→oTrx1→procaspase-3 transnitrosation cascade using a peptide inhibitory of the GC1/Trx1 interaction promotes Jurkat T-cells apoptosis.

### Example 5: Selected Results

The NO-cGMP pathway is a crucial component of vasorelaxation, regulation of blood pressure, cardiac protection, and platelet aggregation. Further, it has been demonstrated herein that the interaction between GC1 and Trx1 supports NO-stimulated GC1 activity, and thus could be essential for cardiovascular homeostasis. On the other hand, it has been recently shown that the formation of a thiol-oxidized SNO-GC1/oTrx1 complex carries transnitrosation cascades. Some of the targets of this oxidized complex are known to be regulated by the NO-cGMP pathway. As oxidative conditions lead to disruption of the canonical NO-GC1-cGMP pathway via heme oxidation and extensive S-nitrosation of GC1, it has been proposed herein that the transnitrosation activity of SNO-GC1, amplified by oTrx1, provides an adaptive response to oxidative stress.

The chemistry of the interaction between Trx1 and GC1 under oxidized and reduced conditions is different. Under reduced conditions, previous mutational analyses showed that the association between GC1 and Trx1 involved a mixed disulfide, potentially between the C³² of Trx1′ reduced active site and C⁶¹⁰ of the α subunit of the GC1 heterodimer. In contrast, under diamide-oxidized conditions, oTrx1 is detected as a dimer, as previously observed. Previous mass spectrometry (MS) analyses indicated that oTrx1′ Cys active site forms a disulfide and C⁷³ is amenable to S-nitrosation, indicating that oTrx1 loses its reductase activity but gains a transnitrosation activity.

The interaction between oTrx1 and SNO-GC1 has been successfully characterized, revealing, a unilateral transfer of S-nitrosothiols from SNO-GC1 to oTrx1 and identifying C⁶¹⁰ of the α subunit as a major “SNO donor” (to the Trx1-C⁷³ SNO acceptor). In this oxidized complex, the interaction with Trx1 could not involve a mixed disulfide with (C3 of Trx1, as (C² is engaged in a disulfide with C³⁵. To gain insight into these various complexes and their relevance, an inhibitory peptide of the interaction was used. This permitted circumvention of the potential activity/conformational alterations generated by mutations of GC1 or Trx1.

The downside of not using Trx1 mutants meant that the ability to “trap” this complex was lost as was analysis via pull-down of this transient complex. The peptides were designed around C⁶¹⁰ of the α subunit to theoretically block both the mixed disulfide of the GC1/Trx1 reduced complex and the S-nitrosothiol transfer between SNO—C⁶¹⁰ and C⁷³ in the oxidized complex.

It has been demonstrated herein in vitro that these C⁶¹⁰-mimetic peptides blunt the Trx1-dependent enhancement of NO-stimulated GC1 activity without affecting directly NO-stimulated GC1 activity, indicating that the peptides did not alter the conformational/structural properties of the NO-responsive GC1 heterodimer. The efficiency of the peptides was confirmed in cells after rendering cell-permeable one selected peptide. It was inferred that the selected peptide (RKINVSPTTYR) (SEQ ID NO:3), which does not contain a disulfide bond unlike targets of Trx1 reductase activity, should not be able to compete with these targets thereby should not interfere with the thiol redox regulatory function of Trx1 and the cellular redox state. On the other hand, C⁶¹⁰ is in a solvent exposed region of the α subunit of GC1 heterodimer and has the potential to interact with other proteins. Without wishing to be bound by any theory, one of these proteins could be a protein disulfide isomerase (PD1), as it has been previously shown that GC1 was interacting with PD1 via a mixed disulfide. The consequences of potentially blocking this interaction or others with the inhibitory peptide will need to be addressed in future studies.

The assays of reductase activity of Trx1 and GC1 in the absence and presence of the inhibitory peptides revealed that GC1 has the ability, in vitro, to reduce the Trx1-oxidized dimer. As this reaction was blocked by the inhibitory peptide, it strongly suggests that the disulfide exchange involves αC⁶¹⁰. Because of the difference in experimental conditions, the Cys engaged in the hornodimer have not been unequivocally identified and could be C⁶⁹, C⁷³ or/and C³² of the active site. As C⁶¹⁰ is involved in a mixed disulfide with C³² of Trx1, and reduction of Trx1-homodimer by GC1 is blocked by the inhibitory peptide, it is hypothesized that C⁶¹¹ could reduce disulfide bonds involving C³² in both the active site (C³²—C³⁵) and homodinerization.

GC1 in vivo is found as dimer that are sensitive to reducing agents and it is proposed that disulfides could have a role in GC1 response to NO. While the diamide-induced multimers of GC1 might not reproduce physiological conditions, the results indicate that rTrx1 can recognize these GC1 multimers and convert them to monomers in an efficient way. Because this Trx1-dependent reduction of GC1 multimers is completely blocked by the inhibitory peptide, this suggests that the multimers formation involves C⁶¹¹ of the α subunit, including a potential C⁶¹¹ disulfide bond between two GC1 heterodimers. In contrast, it has been confirmed herein that rTrx1 cannot reduce thiol oxidation of S-nitrosated GC1 (SNO-GC1). Together these results suggest that rTrx1 is not able to interact with SNO-GC1 in vitro.

On the other hand, it has been confirmed that oTrx1 interacts with SNO-GC1 and demonstrated that the transfer of S-nitrosothiol from SNO-GC1 to oTrx1 is blocked by the inhibitory peptide. The present disclosure describes in vitro identification of other Cys residues of GC1 that drive a nitrosothiols transfer. The bands of biotinylated SNO-GC1 mixed with oTrx1 are undetectable on the blot of FIG. 7A, as expected if there is a complete transfer of nitrosothiols but in the presence of the inhibitory peptide the intensity of SNO-GC1 is similar to the controls SNO-GC1, and SNO-GC1+rTrx1, as if no SNO transfer occurs. This may suggest that the inhibitory peptide blocks the formation of the complex SNO-GC1/oTrx1 rather than the specific SNO-αC⁶¹⁰ to Trx1-C⁷³ transnitrosation reaction.

To test the biological/therapeutic utility of this inhibitory peptide, blocking of GC1/Trx1-dependent transnitrosation under specific pathophysiological conditions was contemplated, with an emphasis on the known link between cancer cell proliferation and inhibition of caspases activity by S-nitrosation. Jurkat T cells, for which Trx1-dependent transnitrosation was shown to inhibit the apoptotic caspase-3 pathway were utilized. It was observed that, in the absence or presence of etoposide (ETO), which induces apoptosis, baseline levels of global S-nitrosation were elevated in controls (DMSO and scramble peptide) and correlated with strong S-nitrosation of procaspase-3 and detectable SNO-Trx1. However, in the presence of the inhibitory peptide, the basal S-nitrosated procaspase-3 levels were drastically reduced and SNO-Trx1 was barely detectable. When ETO was added, SNO-procaspase 3 levels were slightly reduced with the controls and the inhibitory peptide, suggesting that ETO reduces S-nitrosation of procaspase-3 to some extent but not as pronounced as with Fas treatment. Conversely, ETO treatment increased significantly caspase-3 activity; importantly the caspase-3 activity was significantly higher in the cells treated with the inhibitory peptide.

Together with the decreased S-nitrosation of procaspase-3 and Trx1, compared to control DMSO and scramble peptide, the increase in caspase-3 activity indicates that the inhibitory peptide efficiently blocks the transfer of nitrosothiol from GC1 to Trx1 and consequently decreases transnitrosation from Trx1 to procaspase 3. Disruption of the SNO-GC1→Trx1→procaspase3 transnitrosation cascade confirms that SNO-GC1 is a major source of SNO-Trx1 and importantly that this mimetic peptide could be a valuable tool to potentially decrease tumor development associated with excess S-nitrosation. In fact, a direct correlation between Trx1 and S-nitrosation has been established in other cancer types, including hepatocellular carcinoma. Another promising aspect of this peptide is that under these conditions of nitrosative/oxidative stress, SNO-GC1 cannot interact with rTrx1 thus the peptide will not interfere with Trx1 ability to regulate the redox state of the cells, including its denitrosation activity. This could be important considering the dichotomous effect of Trx1 in the development and inhibition of tumor growth in relation to Trx1 ability to transnitrosate and denitrosate various caspases as a function of its redox state. Interestingly, GC1 α subunit, which is sufficient to initiate transnitrosation cascades, was reported to mediate, independently of the 3 subunit or cGMP production, prostate and endometrial cancer cell proliferation. Together, these reports and the present study support the hypothesis that the α subunit of GC1 is a key player in oncogenic processes in association with oTrx1 and transnitrosation reactions.

### Example 6: Distribution of Inhibitory Peptide in Mouse Mesenteric Arteries

Isolated mesenteric arteries (MA) are small resistance blood vessels responsible for the regulation of blood pressure. In order to test the effect of inhibitory peptide on blood vessels, a comparison was done between vessels treated with or without inhibitory peptide.

### Methods

MA were incubated at 37° C. with 400 ng of the inhibitory peptide for 1 h or the solvent DMSO (7%). Inverted fluorescence microscope was used to detect FITC of the peptide. The same exposure time was used for the DMSO and peptide-treated vessels.

### Results

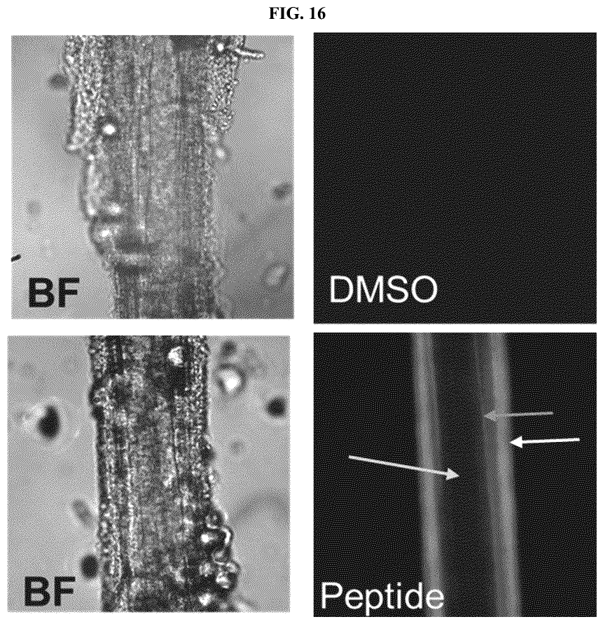

The results show that the mimetic/inhibitory peptide could penetrate the walls of mouse' blood vessels. In addition to being useful for vascular studies of regulation of blood pressure, this indicates that this peptide should penetrate tumors. FIG. 16 shows the results of images taken of the two MA samples. The image indicates efficient uptake of the peptide. The left panels indicate the bright field picture of two MA treated with DMSO (upper left) or the inhibitory peptide (lower left). Inverted fluorescent microscope was used to detect the FITC of the peptide (lower right panel) at 10× magnification (green fluorescence). The yellow arrow indicates the lumen of the vessels; the orange arrow points to the endothelial cell layer and the white arrow to the smooth muscle cell layers. The inhibitory peptide was able to penetrate the walls of mouse' blood vessels. In addition to being useful for our vascular studies of regulation of blood pressure, this is a good indication that this peptide should easily penetrate tumors. Mouse study of blood pressure can further establish the benefit of peptide in vivo.

### Example 7: Characterization of Complex Formation

Peptide biophysical conformation can be studied in vitro for interaction of the peptide with the complex GC1/Trx1 using techniques such as but not limited to Surface Plasmon Resonance, Isothermal Calorimetry and Biolayer Interferometry to conduct competitive binding assays. The detection of the complex formation can be used to optimize the peptide efficiency in disrupting the complex.

In vivo testing of peptide with resistant prostate cancer cells can be used to gauge the role of the GC1/Trx1 complex-dependent S-nitrosation in the development of more aggressive (castration resistance) prostate cancer. The peptide can be used to assess death and proliferation of prostate cancer cells as a function of the maintenance or disruption of the complex.

### Sequence Listing

Peptide 1
SEQ ID NO: 1
VTLANKFESCS
Peptide 2
SEQ ID NO: 2
NKFESCSVPRK
Peptide 3
SEQ ID NO: 3
RKINVSPTTYR
Scramble
SEQ ID NO: 4
KSRVNPSKCEF
FITC-
Inhibitory peptide
SEQ ID NO: 5
FITC-RKKRWFRRRRPKWKKRKINVSPTTYR
(FITC-fluorescein isothiocyanate)
FITC-
Scramble peptide
SEQ ID NO: 6
FITC-RKKRWFRRRRPKWKKKSRVNPSKCEF
(FITC-fluorescein isothiocyanate)
GC1 Site
SEQ ID NO: 7
SVFAGVVGGKMPRYCLFGNNVTLANKFESCSVPR
KINVSPTTYRLLKDCPGFVFT
CPP1
SEQ ID NO: 8
RKKRWFRRRRPKWKK
TAT peptide
SEQ ID NO: 9
YGRKKRRQRRR
penetratin
SEQ ID NO: 10
RQIKIWFQNRRMKWKK
antennapedia peptide
SEQ ID NO: 11
RQIKIWFQNRRMKWKK
transportan
SEQ ID NO: 12
GWTLNSAGYLLGKINLKALAALAKKIL
VP22
SEQ ID NO: 13
RKKRRQRRR
MAP
SEQ ID NO: 14
KLALKLALKALKAALKLA
CADY
SEQ ID NO: 15
CYGALFLGFLGAAGIAKV
Pep-1
SEQ ID NO: 16
KETWWETWWTEWSQPKKKRKV
MPG peptide
SEQ ID NO: 17
GALFLGFLGAAGSTMGAWSQPKKKRKV
TP10
SEQ ID NO: 18
AGYLLGKINLKALAALAKKIL
PEP-NP
SEQ ID NO: 19
HAIYPRHVVGGRRR
SynB1
SEQ ID NO: 20
RRRQRRKKR
TP10-Luc
SEQ ID NO: 21
GLFGALFKAGYLLGKINLKALAALAKKILUC
PepFect6
SEQ ID NO: 22
RQIKIWFQNRRMKWYRWYCKYDK
CADY-B2
SEQ ID NO: 23
CYGALFLGFLGAAGIAKVGGLGC
transportan-10
SEQ ID NO: 24
GWTLNSAGYLLGKINLKALAALAKKIL
SynB3
SEQ ID NO: 25
KKWKMRRNQFWVKVQRG
TAT-PTD4
SEQ ID NO: 26
YGRKKRRQRRRGGGG
Pen-2
SEQ ID NO: 27
RQIKIWFQNRRMKWKKKK
MPG-DHFR
SEQ ID NO: 28
GALFLGFLGAAGSTMGAWSQPKKKRKV-HHHHHHH
MAP-NLS
SEQ ID NO: 29
KLALKLALKALKAALKLAQSARLTTAVARELKV
Pep-3
SEQ ID NO: 30
KETWWETWWTEWSQPKKKRKVGGGC
TP10-VEGF
SEQ ID NO: 31
AGYLLGKINLKALAALAKKILVK
PEP-1-EGFP
SEQ ID NO: 32
KETWWETWWTEWSQPKKKRKV-ENLYFQG
TAT-GFP
SEQ ID NO: 33
YGRKKRRQRRRGFPVAT
human soluble guanylyl cyclase
SEQ ID NO: 34
MFCTKLKDLKITGECPFSLLAPGQVPNESSEEAAGSSESCKATMPI
CQDIPEKNIQESLPQRKTSRSRVYLHTLAESICKLIFPEFERLNV
ALQRTLAKHKIKESRKSLEREDFEKTIAEQAVAAGVPVEVIKESL
GEEVFKICYEEDENILGVVGGTLKDFLNSFSTLLKQSSHCQEAGK
RGRLEDASILCLDKEDDFLHVYYFFPKRTTSLILPGIIKAAAHVL
YETEVEVSLMPPCFHNDCSEFVNQPYLLYSVHMKSTKPSLSPSKP
QSSLVIPTSLFCKTFPFHEMEDKDMTILQFGNGIRRLMNRRDFQG
KPNFEEYFEILTPKINQTFSGIMTMLNMQFVVRVRRWDNSVKKSS
RVMDLKGQMIYIVESSAILELGSPCVDRLEDFTGRGLYLSDIPIH
NALRDVVLIGEQARAQDGLKKRLGKLKATLEQAHQALEEEKKKTV
DLLCSIFPCEVAQQLWQGQVVQAKKFSNVTMLFSDIVGFTAICSQ
CSPLQVITMLNALYTRFDQQCGELDVYKVETIGDAYCVAGGLHKE
SDTHAVQIALMAVKMMELSDEVMSPHGEPIKMRIGLHSGSVFAGV
VGVKMPRYCLFGNNVTLANKFESCSVPRKINVSPTTYRLLKDCPG
FVFTPRSREELPPNFPSEIPGICHFLDAYQQGTNSKPCFQKKDVE
DGNANFLGKASGID

### ENUMERATED EMBODIMENTS

The following exemplary embodiments are provided, the numbering of which is not to be construed as designating levels of importance:

Embodiment 1 provides a construct comprising a polypeptide comprising amino acid residues 581-635 of soluble guanylyl cyclase (GC1) (SEQ ID NO:34), SVFAGVVGGKMPRYCLFGNNVTLANKFESCSVPRKINVSPTTYRLLKDCPGFVFT (SEQ ID NO:7), or a fragment thereof, wherein at least one of the following applies:
(a) the polypeptide is further conjugated to at least one cell penetrating peptide (CPP); and
(b) the polypeptide is further derivatized.

Embodiment 2 provides the construct of Embodiment 1, wherein the derivatization of the polypeptide comprises at least one selected from the group consisting of methylation, amidation, and acetylation.

Embodiment 3 provides the construct of Embodiment 1 or 2, wherein the derivatization of the polypeptide comprises conjugation to a fluorophore, optionally wherein the fluorophore is fluorescein isothiocyanate (FJTC).

Embodiment 4 provides the construct of any one of Embodiments 1-3, wherein the polypeptide comprises a fragment of the amino acid residues 581-635 of GC1.

Embodiment 5 provides the construct of Embodiment 4, wherein the fragment comprises 7, 8, 9, 10, 11, 12, 13, 14, 15, 16, 17, 18, 19, or 20 amino acid residues of the amino acid residues 581-635 of GC1, optionally wherein the fragment comprises 11 amino acid residues.

Embodiment 6 provides the construct of Embodiment 4 or 5, wherein the fragment comprises amino acid residues 601-624 of GC.

Embodiment 7 provides the construct of any one of Embodiments 1-6, wherein the fragment consists essentially of any one of SEQ ID NOs:1-3.

Embodiment 8 provides the construct of any one of Embodiments 1-7, wherein the fragment consists essentially of SEQ ID NO:3.

Embodiment 9 provides the construct of any one of Embodiments 1-8, wherein the at least one CPP shares at least 85% sequence identity with any of SEQ ID NOs:8-33.

Embodiment 10 provides the construct of any one of Embodiments 1-9, wherein the at least one CPP comprises SEQ ID NO:8.

Embodiment 11 provides the construct of any one of Embodiments 1-10, wherein the amino acid residues 581-635 of GC1, or the fragment thereof, are covalently linked to the at least one CPP via the N-terminus amino group of the polypeptide, the C-terminus carboxyl group of the polypeptide, and/or a nucleophilic moiety of an amino acid side chain.

Embodiment 12 provides the construct of Embodiment 11, wherein the amino acid residues 581-635 of GC1, or the fragment thereof, are covalently linked to the CPP via the N-terminus amino group of the polypeptide.

Embodiment 13 provides the construct of Embodiment 11 or 12, wherein the covalent linkage comprises an amide bond.

Embodiment 14 provides the construct of any one of Embodiments 1-13, which shares at least 85% sequence identity with any of SEQ ID NO:5.

Embodiment 15 provides the construct of any one of Embodiments 1-14, which is SEQ ID NO:5.

Embodiment 16 provides a pharmaceutical composition comprising the construct of any one of Embodiments 1-15 and at least one pharmaceutically acceptable carrier.

Embodiment 17 provides a method of treating, preventing, and/or ameliorating a disease or disorder in a subject, the method comprising administering to the subject a therapeutically effective amount of a construct comprising a polypeptide comprising amino acid residues 581-635 of soluble guanylyl cyclase (GC1) (SEQ ID NO:34), SVFAGVVGGKMPRYCLFGNNVTLANKFESCSVPRKINVSPTTYRLLKDCPGFVFT (SEQ ID NO:7), or a fragment thereof.

Embodiment 18 provides the method of Embodiment 17, wherein the polypeptide comprises a fragment of the amino acid residues 581-635 of GC1.

Embodiment 19 provides the method of Embodiment 18, wherein the fragment comprises 7, 8, 9, 10, 11, 12, 13, 14, 15, 16, 17, 18, 19, or 20 amino acid residues of the amino acid residues 581-635 of GC1, optionally wherein the fragment comprises 11 amino acid residues.

Embodiment 20 provides the method of any one of Embodiments 17-19, wherein the fragment comprises amino acid residues 601-624 of GC1.

Embodiment 21 provides the method of any one of Embodiments 17-20, wherein the fragment consists essentially of any one of SEQ ID NOs:1-3.

Embodiment 22 provides the method of any one of Embodiments 17-21, wherein the fragment consists essentially of SEQ ID NO:3.

Embodiment 23 provides the method of any one of Embodiments 17-22, wherein the polypeptide is conjugated to at least one cell penetrating peptide (CPP).

Embodiment 24 provides the method of Embodiment 23, wherein the at least one CPP shares at least 85% sequence identity with any of SEQ ID NOs:8-33.

Embodiment 25 provides the method of Embodiment 23 or 24, wherein the at least one CPP comprises SEQ ID NO:8.

Embodiment 26 provides the method of any one of Embodiments 23-25, wherein the amino acid residues 581-635 of GC1, or the fragment thereof, are covalently linked to the at least one CPP via the N-terminus amino group of the polypeptide, a C-terminus amino group of the polypeptide, and/or a nucleophilic moiety of an amino acid side chain.

Embodiment 27 provides the method of Embodiment 26, wherein the amino acid residues 581-635 of GC1, or the fragment thereof, are covalently linked to the CPP via the N-terminus amino group of the polypeptide.

Embodiment 28 provides the method of Embodiment 26 or 27, wherein the covalent linkage comprises an amide bond.

Embodiment 29 provides the method of any one of Embodiments 17-28, wherein at least one amino acid residue of the polypeptide is derivatized.

Embodiment 30 provides the method of Embodiment 29, wherein the derivatization comprises at least one selected from the group consisting of methylation, amidation, and acetylation.

Embodiment 31 provides the method of Embodiment 29 or 30, wherein the derivatization comprises conjugation to a fluorophore, optionally wherein the fluorophore is fluorescein isothiocyanate (FITC).

Embodiment 32 provides the method of any one of Embodiments 17-31, wherein the construct shares at least 85% sequence identity with any of SEQ ID NO:5.

Embodiment 33 provides the method of any one of Embodiments 17-32, wherein the construct is SEQ ID NO:5.

Embodiment 34 provides the method of any one of Embodiments 17-33, wherein the construct is administered as a pharmaceutical composition.

Embodiment 35 provides the method of any one of Embodiments 17-34, wherein formation of a GC1/Trx1 complex is inhibited.

Embodiment 36 provides the method of any one of Embodiments 17-35, wherein the disease or disorder comprises cancer.

Embodiment 37 provides the method of Embodiment 36, wherein the cancer is of the prostate, lymphatic system, bladder, blood, bone, bone marrow, brain, breast, colon, esophagus, gastrointestine, gum, head, kidney, liver, lung, nasopharynx, neck, ovary, skin, stomach, testis, tongue, and/or uterus.

Embodiment 38 provides the method of any one of Embodiments 17-35, wherein disease or disorder is a blood pressure disease or disorder.

Embodiment 39 provides the method of Embodiment 38, wherein the blood pressure disease or disorder which is selected from the group consisting of hypertension, hypotension, and cardiac dysfunction.

Embodiment 39 provides the method of any one of Embodiments 17-39, wherein the subject is a mammal.

Embodiment 40 provides the method of Embodiment 40, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A construct comprising a polypeptide comprising amino acid residues 581-635 of soluble guanylyl cyclase (GC1) (SEQ ID NO:34), SVFAGVVGGKMPRYCLFGNNVTLANKFESCSVPRKINVSPTTYRLLKDCPGFVFT (SEQ ID NO:7), or a fragment thereof, wherein at least one of the following applies:
(a) the polypeptide is further conjugated to at least one cell penetrating peptide (CPP); and
(b) the polypeptide is further derivatized.

2. The construct of claim 1,
wherein the derivatization of the polypeptide comprises at least one selected from the group consisting of methylation, amidation, and acetylation; or
wherein the derivatization of the polypeptide comprises conjugation to a fluorophore, optionally wherein the fluorophore is fluorescein isothiocyanate (FITC).

3. The construct of claim 1, wherein at least one of the following applies:
(a) the polypeptide comprises a fragment of the amino acid residues 581-635 of GC1, optionally wherein the fragment comprises amino acid residues 601-624 of GC1;
(b) the fragment comprises 7, 8, 9, 10, 11, 12, 13, 14, 15, 16, 17, 18, 19, or 20 amino acid residues of the amino acid residues 581-635 of GC1, optionally wherein the fragment comprises 11 amino acid residues;
(c) the fragment comprises 7, 8, 9, 10, 11, 12, 13, 14, 15, 16, 17, 18, 19, or 20 amino acid residues of the amino acid residues 581-635 of GC1, optionally wherein the fragment comprises 11 amino acid residues.

4. The construct of claim 1, wherein the fragment consists essentially of any one of SEQ ID NOs:1-3; optionally wherein the fragment consists essentially of SEQ ID NO:3.

5. The construct of claim 1, wherein the at least one CPP shares at least 85% sequence identity with any of SEQ ID NOs:8-33; optionally wherein the at least one CPP comprises SEQ ID NO:8.

6. The construct of claim 1, wherein at least one of the following applies:
(a) the amino acid residues 581-635 of GC1, or the fragment thereof, are covalently linked to the at least one CPP via the N-terminus amino group of the polypeptide, the C-terminus carboxyl group of the polypeptide, or a nucleophilic moiety of an amino acid side chain, optionally wherein the covalent linkage comprises an amide bond;
(b) the amino acid residues 581-635 of GC1, or the fragment thereof, are covalently linked to the CPP via the N-terminus amino group of the polypeptide;
(c) the construct shares at least 85% sequence identity with any of SEQ ID NO:5, optionally wherein the construct is SEQ NO: 5.

7. A pharmaceutical composition comprising the construct of claim 1 and at least one pharmaceutically acceptable carrier.

8. A method of treating, preventing, and/or ameliorating a disease or disorder in a subject, the method comprising administering to the subject a therapeutically effective amount of a construct comprising a polypeptide comprising amino acid residues 581-635 of soluble guanylyl cyclase (GC1) (SEQ ID NO:34), SVFAGVVGGKMPRYCLFGNNVTLANKFESCSVPRKINVSPTTYRLLKDCPGFVFT (SEQ ID NO:7), or a fragment thereof.

9. The method of claim 8, wherein the polypeptide comprises a fragment of the amino acid residues 581-635 of GC1; optionally wherein the fragment comprises 7, 8, 9, 10, 11, 12, 13, 14, 15, 16, 17, 18, 19, or 20 amino acid residues of the amino acid residues 581-635 of GC1, optionally wherein the fragment comprises 11 amino acid residues.

10. The method of claim 8, wherein at least one of the following applies:
(a) the fragment comprises amino acid residues 601-624 of GC1;
(b) the fragment consists essentially of any one of SEQ ID NOs:1-3; or
(c) the polypeptide is conjugated to at least one cell penetrating peptide (CPP).

11. The method of claim 8, wherein the fragment consists essentially of SEQ ID NO:3.

12. The method of claim 8, wherein the at least one CPP shares at least 85% sequence identity with any of SEQ ID NOs:8-33, optionally wherein the at least one CPP comprises SEQ ID NO:8.

13. The method of claim 8, wherein the amino acid residues 581-635 of GC1, or the fragment thereof, are covalently linked to the at least one CPP via the N-terminus amino group of the polypeptide, a C-terminus amino group of the polypeptide, or a nucleophilic moiety of an amino acid side chain, optionally wherein the amino acid residues 581-635 of GC1, or the fragment thereof, are covalently linked to the CPP via the N-terminus amino group of the polypeptide.

14. The method of claim 13, wherein at least one of the following applies:
(a) the covalent linkage comprises an amide bond; or
(b) at least one amino acid residue of the polypeptide is derivatized.

15. The method of claim 14,
wherein the derivatization comprises at least one selected from the group consisting of methylation, amidation, and acetylation; or
wherein the derivatization comprises conjugation to a fluorophore, optionally wherein the fluorophore is fluorescein isothiocyanate (FITC).

16. The method of claim 8, wherein the construct shares at least 85% sequence identity with any of SEQ ID NO:5, optionally wherein the construct comprise the amino acid sequence of SEQ ID NO:5.

17. The method of claim 8, wherein the construct is administered as a pharmaceutical composition, optionally wherein the administering inhibits formation of a GC1/Trx1 complex.

18. The method of claim 8, wherein the disease or disorder comprises cancer; optionally wherein the cancer is of the prostate, lymphatic system, bladder, blood, bone, bone marrow, brain, breast, colon, esophagus, gastrointestine, gum, head, kidney, liver, lung, nasopharynx, neck, ovary, skin, stomach, testis, tongue, or uterus.

19. The method of claim 8, wherein disease or disorder is a blood pressure disease or disorder, optionally wherein the blood pressure disease or disorder is selected from the group consisting of hypertension, hypotension, and cardiac dysfunction.

20. The method of claim 8, wherein the subject is a mammal, optionally wherein the mammal is a human.


SEQUENCE LISTING (34 sequences)

SEQ ID NO: 1 - protein, 11 aa, synthetic construct
VTLANKFESCS

SEQ ID NO: 2 - protein, 11 aa, synthetic construct
NKFESCSVPRK

SEQ ID NO: 3 - protein, 11 aa, synthetic construct
RKINVSPTTYR

SEQ ID NO: 4 - protein, 11 aa, synthetic construct
KSRVNPSKCEF

SEQ ID NO: 5 - protein, 26 aa, synthetic construct
  SITE 1: note = NH-FITC
RKKRWFRRRRPKWKKRKINVSPTTYR

SEQ ID NO: 6 - protein, 26 aa, synthetic construct
  SITE 1: note = NH-FITC
RKKRWFRRRRPKWKKKSRVNPSKCEF

SEQ ID NO: 7 - protein, 55 aa, synthetic construct
SVFAGVVGGKMPRYCLFGNNVTLANKFESCSVPRKINVSPTTYRLLKDCPGFVFT

SEQ ID NO: 8 - protein, 15 aa, synthetic construct
RKKRWFRRRRPKWKK

SEQ ID NO: 9 - protein, 11 aa, synthetic construct
YGRKKRRQRRR

SEQ ID NO: 10 - protein, 16 aa, synthetic construct
RQIKIWFQNRRMKWKK

SEQ ID NO: 11 - protein, 16 aa, synthetic construct
RQIKIWFQNRRMKWKK

SEQ ID NO: 12 - protein, 27 aa, synthetic construct
GWTLNSAGYLLGKINLKALAALAKKIL

SEQ ID NO: 13 - protein, 9 aa, synthetic construct
RKKRRQRRR

SEQ ID NO: 14 - protein, 18 aa, synthetic construct
KLALKLALKALKAALKLA

SEQ ID NO: 15 - protein, 18 aa, synthetic construct
CYGALFLGFLGAAGIAKV

SEQ ID NO: 16 - protein, 21 aa, synthetic construct
KETWWETWWTEWSQPKKKRKV

SEQ ID NO: 17 - protein, 27 aa, synthetic construct
GALFLGFLGAAGSTMGAWSQPKKKRKV

SEQ ID NO: 18 - protein, 21 aa, synthetic construct
AGYLLGKINLKALAALAKKIL

SEQ ID NO: 19 - protein, 14 aa, synthetic construct
HAIYPRHVVGGRRR

SEQ ID NO: 20 - protein, 9 aa, synthetic construct
RRRQRRKKR

SEQ ID NO: 21 - protein, 28 aa, synthetic construct
GLFGALFKAGYLLGKINLKALAALAKKI

SEQ ID NO: 22 - protein, 23 aa, synthetic construct
RQIKIWFQNRRMKWYRWYCKYDK

SEQ ID NO: 23 - protein, 23 aa, synthetic construct
CYGALFLGFLGAAGIAKVGGLGC

SEQ ID NO: 24 - protein, 27 aa, synthetic construct
GWTLNSAGYLLGKINLKALAALAKKIL

SEQ ID NO: 25 - protein, 17 aa, synthetic construct
KKWKMRRNQFWVKVQRG

SEQ ID NO: 26 - protein, 15 aa, synthetic construct
YGRKKRRQRRRGGGG

SEQ ID NO: 27 - protein, 18 aa, synthetic construct
RQIKIWFQNRRMKWKKKK

SEQ ID NO: 28 - protein, 34 aa, synthetic construct
GALFLGFLGAAGSTMGAWSQPKKKRKVHHHHHHH

SEQ ID NO: 29 - protein, 33 aa, synthetic construct
KLALKLALKALKAALKLAQSARLTTAVARELKV

SEQ ID NO: 30 - protein, 25 aa, synthetic construct
KETWWETWWTEWSQPKKKRKVGGGC

SEQ ID NO: 31 - protein, 23 aa, synthetic construct
AGYLLGKINLKALAALAKKILVK

SEQ ID NO: 32 - protein, 28 aa, synthetic construct
KETWWETWWTEWSQPKKKRKVENLYFQG

SEQ ID NO: 33 - protein, 17 aa, synthetic construct
YGRKKRRQRRRGFPVAT

SEQ ID NO: 34 - protein, 690 aa, synthetic construct
MFCTKLKDLKITGECPFSLLAPGQVPNESSEEAAGSSESCKATMPICQDIPEKNIQESLPQRKTSRSRVYLHTLAESICKLIFPEFERLNVALQRTLAKHKIKESRKSLEREDFEKTIAEQAVAAGVPVEVIKESLGEEVFKICYEEDENILGVVGGTLKDFLNSFSTLLKQSSHCQEAGKRGRLEDASILCLDKEDDFLHVYYFFPKRTTSLILPGIIKAAAHVLYETEVEVSLMPPCFHNDCSEFVNQPYLLYSVHMKSTKPSLSPSKPQSSLVIPTSLFCKTFPFHFMFDKDMTILQFGNGIRRLMNRRDFQGKPNFEEYFEILTPKINQTFSGIMTMLNMQFVVRVRRWDNSVKKSSRVMDLKGQMIYIVESSAILFLGSPCVDRLEDFTGRGLYLSDIPIHNALRDVVLIGEQARAQDGLKKRLGKLKATLEQAHQALEEEKKKTVDLLCSIFPCEVAQQLWQGQVVQAKKFSNVTMLFSDIVGFTAICSQCSPLQVITMLNALYTRFDQQCGELDVYKVETIGDAYCVAGGLHKESDTHAVQIALMAVKMMELSDEVMSPHGEPIKMRIGLHSGSVFAGVVGVKMPRYCLFGNNVTLANKFESCSVPRKINVSPTTYRLLKDCPGFVFTPRSREELPPNFPSEIPGICHFLDAYQQGTNSKPCFQKKDVEDGNANFLGKASGID